(19) United States
(12) Patent Application Publication
(10) Pub. No.: US 2023/0096096 A1
(43) Pub. Date: Mar. 30, 2023

(54) MARROW ASPIRATION DEVICE AND METHOD OF USE

(71) Applicant: Royal Biologics, Hackensack, NJ (US)

(72) Inventors: Darin EWER, Providence, UT (US); Salvatore A. LEO, Miami, FL (US)

(73) Assignee: Royal Biologics, Hackensack, NJ (US)

(21) Appl. No.: 17/485,741

(22) Filed: Sep. 27, 2021

(51) Int. Cl. A61M 1/00; A61B 17/34

(52) U.S. Cl. CPC A61M 1/815; A61B 17/3472; A61M 2202/10; A61B 2017/00407

(57) ABSTRACT

A bone marrow aspiration device may include a housing having a proximal end and a distal end; a cannula, insertable into a tissue, extending from the distal end; a ratcheting mechanism coupled to the housing; a reservoir configured to receive a fluid; an actuator operatively coupled to the ratcheting mechanism and to a distal surface of the bone marrow aspiration device such that user-actuation of the actuator actuates the ratcheting mechanism to draw fluid through the cannula, into the housing, and into the reservoir, and retracts the cannula relative to the distal surface.

### TECHNICAL FIELD

The present disclosure generally relates to the field of medical technology. The present disclosure more specifically relates to the field of bone marrow aspiration from a patient. The disclosed technology includes a marrow aspiration device and method of using the marrow aspiration device to draw a metered amount of marrow from a patient.

### BACKGROUND

Bone marrow is spongy substance that forms within the inner portions of a human bone. This marrow is used by the body to manufacture bone marrow stem cells as well as other substances used to produce blood cells within the body. Developments have occurred in the medical field that allow medical professionals to use this bone marrow for a range of medical procedures such as bone marrow transplants from a bone marrow donor to a recipient. In some cases, the transplanted bone marrow may be used by a patient suffering from a type of cancer that may or may not be bone marrow born. Other medical procedures may also be conducted with the received bone marrow including biopsies and the like to determine the health of a patient.

For this reason, this bone marrow may be harvested from a patient. In some cases, this harvesting of bone marrow is done by inserting a needle into a bone such as a pelvic bone while the patient is under anesthesia. Once a hole is created in both the fat, muscular, and bone tissue, the bone marrow may be accessed and extracted using some extraction such as a syringe. However, due to the patient being under general anesthesia and the potential risks involved in the extraction of the bone marrow, it is necessary to complete the extraction as soon as possible and with the least amount of trauma placed on the patient/donor.

### SUMMARY OF THE INVENTION

The various systems and methods of the present invention have been developed in response to the present state of the art, and in particular, in response to the problems and needs in the art that have not yet been fully solved by currently available marrow aspiration systems and methods.

According to one embodiment, a bone marrow aspiration device may be provided. The bone marrow aspiration device may have a housing having a proximal end and a distal end, a cannula, insertable into a tissue, extending from the distal end, a ratcheting mechanism coupled to the housing, a reservoir configured to receive a fluid, and an actuator. The actuator may be operatively coupled to the ratcheting mechanism and to a distal surface of the bone marrow aspiration device such that user-actuation of the actuator actuates the ratcheting mechanism to draw fluid through the cannula, into the housing, and into the reservoir, and retracts the cannula relative to the distal surface.

The reservoir may have a syringe with a plunger.

The bone marrow aspiration device may further have a multi-slot plunger puller operatively and selectively coupled to the plunger of the syringe. User-actuation of the actuator may cause the multi-slot plunger puller to move distally, creating a negative pressure within the syringe and drawing the fluid into the syringe.

The bone marrow aspiration device may further have a sharp stylet that is insertable into the cannula to allow the distal end of the cannula to penetrate through the tissue.

The bone marrow aspiration device may further have an aspiration cannula that is insertable into the cannula. The aspiration cannula may have aspiration cannula fenestrations that align with cannula fenestrations formed in the cannula when the aspiration cannula is correctly seated, coaxially, within the cannula.

The aspiration cannula may have an O-ring that fluidically seals the aspiration cannula against an inner surface of the housing to create a sealed fluidic path along the aspiration cannula and to the reservoir.

The bone marrow aspiration device may further have a ratcheting switch that is operable to engage the ratcheting mechanism such that user-actuation of the actuator actuates the ratcheting mechanism when the ratcheting switch is in a first position, and disengage the ratcheting mechanism when the ratcheting switch is in a second position such that the distal surface may move independently of the user-actuation of the ratcheting mechanism.

The bone marrow aspiration device may further have a depth stop extension operatively fixed to the distal surface to abut a surface of a tissue and, during a percutaneous incision of the cannula into the tissue, maintain a distance between the surface of the tissue and the distal end.

According to one embodiment, an aspiration device may be provided. The aspiration device may have a housing having a proximal end and a distal end, a cannula, insertable into a tissue, extending from the distal end, a ratcheting mechanism coupled to the housing, and an actuator. The actuator may be operatively coupled to the ratcheting mechanism such that user-actuation of the actuator actuates the ratcheting mechanism to draw fluid through the cannula, into the housing, and into a removeable syringe fluidically coupled to a proximal end of the cannula.

The aspiration device may further have a multi-slot plunger puller operatively and selectively coupled to a plunger of the removeable syringe. User-actuation of the actuator may cause the multi-slot plunger puller to move distally, creating a negative pressure within the syringe and drawing the fluid into the removeable syringe.

The aspiration device may further have an aspiration cannula that is insertable into the cannula. The aspiration cannula may have aspiration cannula fenestrations that align with cannula fenestrations formed in the cannula when the aspiration cannula is correctly seated, coaxially, within the cannula.

The aspiration device may further have a reservoir configured to receive the fluid. The aspiration cannula may have an O-ring that fluidically seals the aspiration cannula against an inner surface of the housing and creates a sealed fluidic path along the aspiration cannula and to the reservoir.

The aspiration device may further have a ratcheting switch that is operable to engage the ratcheting mechanism such that user-actuation of the actuator actuates the ratcheting mechanism when the ratcheting switch is in a first position, and disengage the ratcheting mechanism when the ratcheting switch is in a second position such that a distal surface of the aspiration device may move independently of the user-actuation of the ratcheting mechanism.

The aspiration device may further have a depth stop extension operatively fixed to a distal surface of the aspiration device to abut a surface of a tissue and, during a percutaneous incision of the cannula into the tissue, maintain a distance between the surface of the tissue and the distal end.

According to one embodiment, a bone marrow aspiration device may be provided. The bone marrow aspiration device may have a housing having a proximal end and a distal end, a cannula, insertable into a tissue, extending from the distal end, a removeable syringe fluidically coupled to a proximal end of the cannula at a proximal end of the housing, and an actuator operatively coupled to the syringe such that user-actuation of the actuator draws bone marrow through the cannula, through the housing, and into a proximal end of the removeable syringe.

The bone marrow aspiration device of may further have a multi-slot plunger puller operatively and selectively coupled to a plunger of the removeable syringe. User-actuation of the actuator may cause the multi-slot plunger puller to move distally, creating a negative pressure within the syringe and drawing the bone marrow into the removeable syringe.

The bone marrow aspiration device may further have a ratcheting switch that is operable to engage a ratcheting mechanism such that user-actuation of the actuator actuates the ratcheting mechanism when the ratcheting switch is in a first position, and disengage the ratcheting mechanism when the ratcheting switch is in a second position such that a distal surface of the bone marrow aspiration device may move independent of the user-actuation of the ratcheting mechanism.

The bone marrow aspiration device may further have a depth stop extension operatively fixed to a distal surface of the bone marrow aspiration device to abut a surface of a tissue and, during a percutaneous incision of the cannula into the tissue, maintain a distance between the surface of the tissue and the distal end.

The bone marrow aspiration device may further have a sharp stylet that is insertable into the cannula to allow the distal end of the cannula to penetrate through the tissue.

The bone marrow aspiration device may further have an aspiration cannula that is insertable into the cannula. The aspiration may have aspiration cannula fenestrations that align with cannula fenestrations formed in the cannula when the aspiration cannula is correctly seated, coaxially, within the cannula.

These and other features and advantages of the present invention will become more fully apparent from the following description and appended claims, or may be learned by the practice of the invention as set forth hereinafter.

### BRIEF DESCRIPTION OF THE DRAWINGS

Exemplary embodiments of the invention will become more fully apparent from the following description and appended claims, taken in conjunction with the accompanying drawings. Understanding that these drawings depict only exemplary embodiments and are, therefore, not to be considered limiting of the invention's scope, the exemplary embodiments of the invention will be described with additional specificity and detail through use of the accompanying drawings in which:

FIG. 1 is a side perspective view of a fluid aspiration device according to an embodiment of the present disclosure;

FIG. 2 is a side view of a fluid aspiration device including a removeable syringe according to an embodiment of the present disclosure;

FIG. 3 is a side view of a fluid aspiration device including a removeable syringe and a depth stop according to an embodiment of the present disclosure;

FIG. 4 is a side perspective view of a depth stop according to an embodiment of the present disclosure;

FIG. 5 is a side view of a fluid aspiration device including a removeable syringe, a depth stop, and a stylet according to an embodiment of the present disclosure;

FIG. 6 is a side view of a fluid aspiration device including a removeable syringe, a depth stop, and an aspiration cannula according to an embodiment of the present disclosure;

FIG. 7 is a side view of a sharp stylet according to an embodiment of the present o disclosure;

FIG. 8 is a side view of an aspiration cannula according to an embodiment of the present disclosure;

FIG. 9 is a side perspective view of a ratcheting system of the fluid aspiration device according to an embodiment of the present disclosure;

FIG. 10 is a side view of a fluid aspiration device with a syringe to be operatively coupled to the fluid aspiration device according to an embodiment of the present disclosure;

FIG. 11 is a front perspective view of a fluid aspiration device with a syringe operatively coupled thereto according to an embodiment of the present disclosure;

FIG. 12 is a side view of a fluid aspiration device depicting the introduction of a sharp stylet into the fluid aspiration device according to an embodiment of the present disclosure;

FIG. 13 is a side view of a fluid aspiration device with a syringe and a sharp stylet being inserted into a tissue according to an embodiment of the present disclosure;

FIG. 14 is a side view of a fluid aspiration device with a syringe and an aspiration cannula being inserted into a tissue according to an embodiment of the present disclosure;

FIG. 15 is a side view of a fluid aspiration device with a syringe and an aspiration cannula being inserted into a tissue and resulting actuation of a ratcheting mechanism according to an embodiment of the present disclosure;

FIG. 16 is a side view of a fluid aspiration device with a syringe, an aspiration cannula, and a depth stop being inserted into a tissue according to an embodiment of the present disclosure.

### DETAILED DESCRIPTION

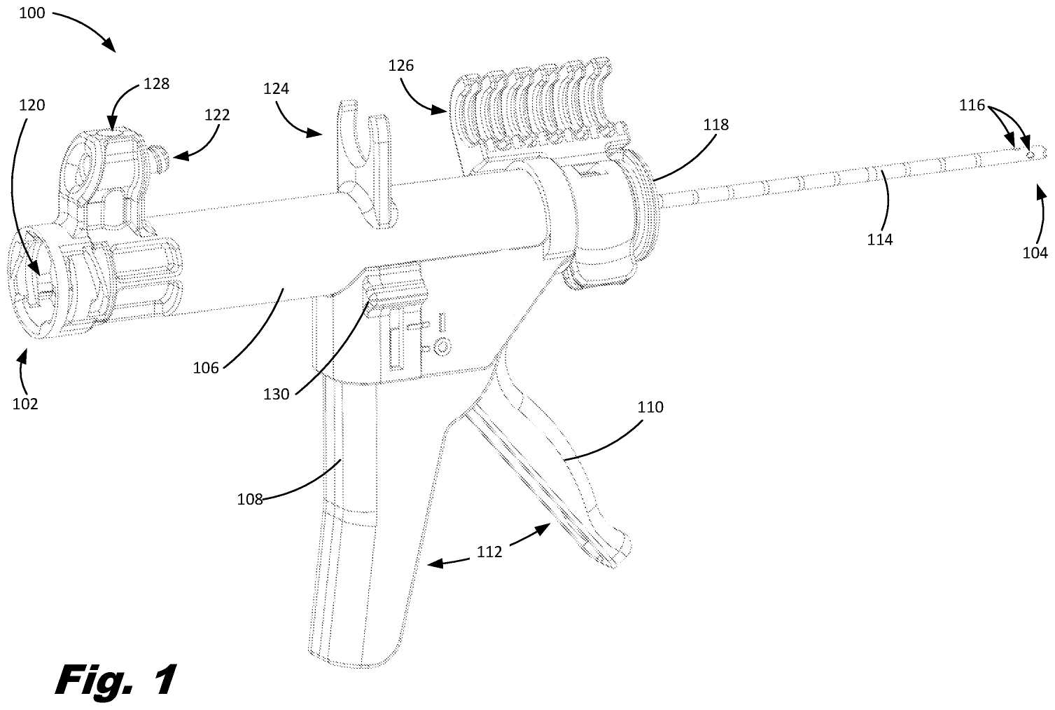

Exemplary embodiments of the invention will be best understood by reference to the drawings, wherein like parts are designated by like numerals throughout. It will be readily understood that the components of the invention, as generally described and illustrated in the Figures herein, could be arranged and designed in a wide variety of different configurations. Thus, the following more detailed description of the embodiments of the apparatus, system, and method, as represented in FIGS. 1A through 9, is not intended to limit the scope of the invention, as claimed, but is merely representative exemplary of exemplary embodiments of the invention.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Bone marrow may be aspirated from within a bone of a patient for a number of reasonings. These reasons may include the biopsy of that marrow in order to diagnose and subsequently treat a disease, biopsy of the marrow in order to determine a stage or progression of a disease, determine whether appropriate levels of constituents (e.g., iron) are present in the marrow, monitor an on-going treatment of a disease, investigate a fever of unknown origin, or as a transplant for a second patient or the original patient.

The process of extracting the bone marrow, called aspiration, may include placing a patient under general anesthesia in an operating room, accessing the bone marrow with a needled syringe, and drawing an amount of bone marrow from the patient. In the case where the patient is the recipient of the bone marrow and is conditioned prior to the procedure using chemotherapy, this process may prove uncomfortable for the patient for many days. In light of these procedures, the pain suffered by the conditioning and the aspiration process under general anesthesia. The aspiration process may take as long as two hours using this process.

In order to decrease the aspiration time and reduce the medical trauma experienced by the patient, the present specification describes a bone marrow aspiration device used to draw a metered amount of bone marrow from the patient. In an embodiment, the bone marrow aspiration device may include a housing having a proximal end and a distal end. In the present specification and in the appended claims, the term “proximal” means a location on the bone marrow aspiration device closest to the user. Additionally, in the present specification and in the appended claims, the term “distal” means a location on the bone marrow aspiration device furthest from the user. The housing of the bone marrow aspiration device may house a cannula and a ratcheting mechanism. The cannula may extend distally from the housing and is insertable into the tissue of a patient. In an embodiment, a sharp stylet and an aspiration cannula may each be selectively inserted, coaxially, into the cannula during use of the bone marrow aspiration device.

The bone marrow aspiration device may also include, in an embodiment, a reservoir configured to receive a fluid such as the bone marrow described herein. Thus, although the present specification describes the use of the bone marrow aspiration device as a device used to aspirate bone marrow, the present specification further contemplates that the bone marrow aspiration device may be used to aspirate any type of fluid from within a patient's body. In an embodiment, the reservoir may be a syringe that includes a removeable plunger.

The bone marrow aspiration device may further include an actuator operatively coupled to the ratcheting mechanism and to a distal surface of the bone marrow aspiration device such that user-actuation of the actuator actuates the ratcheting mechanism to draw fluid through the cannula, into the housing, and into the reservoir, and retracts the cannula relative to the distal surface, for example, by moving the distal surface distally towards the tissue.

In the embodiment where the reservoir is a syringe with a plunger, the bone marrow aspiration device may further include a multi-slot plunger puller operatively and selectively coupled to the plunger of the syringe. During operation, user-actuation of the actuator causes the multi-slot plunger puller to move distally creating a negative pressure within the syringe and drawing the fluid into the syringe. This allows for a metered amount of fluid to be received within the reservoir.

The embodiments also describe the use of a sharp stylet to be placed coaxially within the cannula of the bone marrow aspiration device. The sharp stylet is used by the user to help facilitate that penetration of the cannula into the tissue. This tissue may include skin, fat, muscle, and bone. Once inserted, that user may remove the sharp stylet from within the cannula and replace the sharp stylet for an aspiration cannula that also is placed coaxially within the cannula of the bone marrow aspiration device. In an embodiment, the aspiration cannula may include one or more fenestrations used to allow bone marrow to enter into a hollow portion within the aspiration cannula. When the aspiration cannula is inserted into the cannula, these aspiration cannula fenestrations may align with a number of fenestrations formed in the cannula of the bone marrow aspiration device. Alignment of these fenestrations may occur when the aspiration cannula is seated within the bone marrow aspiration device via a locking mechanism formed at a proximal end of the bone marrow aspiration device. In an embodiment, the aspiration cannula may include an O-ring used to fluidically seal the fluidic path formed by the aspiration cannula, cannula, and fluid reservoir.

FIG. 1 illustrates a side perspective view of an aspiration device 100 according to an embodiment of the present disclosure. The aspiration device 100 may be a fluid aspiration device that pulls an amount of fluid into a fluid reservoir operably couplable to the aspiration device 100. In the context of the present specification, the aspiration device 100 is described as a bone marrow aspiration device 100 used to draw an amount of bone marrow from within a bone. However, although the use of the aspiration device 100 in the context of drawing bone marrow from a bone, the present specification contemplates that the aspiration device 100 may be used for a number of fluids and the example uses described herein are meant as an example use only. The aspiration device 100 may be referred herein as a “bone marrow aspiration device,” a “aspiration device 100,” and a “fluid aspiration device” without limiting the use of the aspiration device 100.

The aspiration device 100 may have a proximal end 102 and a distal end 104. As described herein, the proximal end 102 refers to the end of the aspiration device 100 that is close to the user during use. Additionally, the distal end 104 refers to the end of the aspiration device 100 that is away from the user during use. At the most extreme proximal end 102 end of the aspiration device 100, an instrument port 120 is formed to receive one of a sharp stylet and an aspiration cannula as described herein. At the extreme distal end 104 of the aspiration device 100 is a terminal end of a cannula 114 with the cannula 114 extending into a portion of the housing 106 a distance.

In order to handle the aspiration device 100, a handle 108 may be formed and extend radially from a central axis of the housing 106. The handle may be ergonomically formed to fit the size of a user's hand. To facilitate the holding of the aspiration device 100, the aspiration device 100 may further include an actuator 110. The user may then grip the aspiration device 100 by placing the handle 108 in the user's palm with the user's fingers wrapped around the actuator 110. As such the user may, during use, actuate the actuator 110 by moving it in the direction of the actuator movement 112 line as indicated in FIG.1. Here the user may draw his/her fingers towards the handle 108 in order to actuate the actuator 110. The actuation of the actuator 110 is described in more detail herein.

The cannula 114, along with other instruments like the aspiration cannula described herein, may form a fluidic path from the distal end of the cannula 114 to a fluid reservoir port 122. The fluid reservoir port 122 may receive a fluid reservoir into which bone marrow or other fluid is passed into during use. As described in more detail herein, the fluid reservoir may be a syringe. In this example embodiment, the syringe may include a locking feature such as a series of threads that engage with a counterpart locking feature (e.g., threads) at the fluid reservoir port 122 to fluidically seal the fluid reservoir to the fluidic channel formed within the aspiration device 100. Although the present specification describes the fluid reservoir as a syringe, other types of fluid reservoirs may be used and the example of a syringe is presented to describe the functioning of the aspiration device 100.

In an embodiment, the cannula 114 may include one or more cannula fenestrations 116 formed at a distal end of the cannula 114. These cannula fenestrations 116 may be used to draw bone marrow into the cannula 114, through the fluid reservoir port 122, and into a fluid reservoir. As described herein, an aspiration cannula may be placed coaxially within the cannula 114 and may also have a number of cannulas formed therein (“aspiration cannula fenestrations”). When the aspiration cannula is placed within the cannula 114, the aspiration cannula fenestrations and the cannula fenestrations 116 may match up to allow bone marrow to be drawn into the aspiration cannula and the cannula 114 when the aspiration device 100 is used. This is described in more detail herein.

In the example embodiment where the fluid reservoir is a syringe, the syringe may include a plunger. During operation, the plugger may engage with the aspiration device 100 via a multi-slot plunger puller 126. The multi-slot plunger puller 126 may be moved radially about a central axis of the housing 106 in order to engage the plunger of the syringe. When engaged, the multi-slot plunger puller 126 may pull the plunger as the user actuates the actuator 110. This is accomplished by a ratcheting mechanism formed within the housing 106 of the aspiration device 100 that concurrently advances the multi-slot plunger puller 126 distally and a connected patient o contacting surface 118. The functioning of the ratcheting mechanism in connection with the fluid reservoir, the patient contacting surface 118, and the multi-slot plunger puller 126 is described in more detail herein.

The aspiration device 100 may further include a fluid reservoir mount 124. The fluid reservoir mount 124, in the embodiment shown in FIG. 1 may receive the syringe and plunger and hold the syringe in place during operation of the aspiration device 100 by the user. The present specification contemplates that some other types of devices may be used in place of or in addition to the fluid reservoir mount 124 shown in FIG. 1.

The aspiration device 100, in an embodiment, may also include a plug 128 formed at a proximal end 102 of the aspiration device 100 where the fluidic path created by the cannula 114 meets with the fluid reservoir port 122. This plug 128 may be a permeable membrane made of a rubber or other self-healing material. The self-healing material that the plug 128 is made of may allow for the penetration of a needle into the plug 128 in order to introduce a substance into the collected fluid within the fluid reservoir. This substance may include a stabilizing chemical that preserved the fluid collected during use of the aspiration device 100.

The aspiration device 100 may further include a ratcheting switch 130. The ratcheting switch 130 may engage the ratcheting mechanism, when in a first position, such that user-actuation of the actuator actuates the ratcheting mechanism. The ratcheting switch 130 may disengage the ratcheting mechanism, when the ratcheting switch is in a second position, such that the patient contacting surface 118 may move independent of the user-actuation of the ratcheting mechanism. Here, the disengagement of the ratcheting mechanism allows a user to disengage the multi-slot plunger puller 126 with a plunger of the syringe, move the multi-slot plunger puller 126 and the patient contacting surface 118, and reengage the multi-slot plunger puller 126 with the plunger in order to provide more moving distance for the plunger.

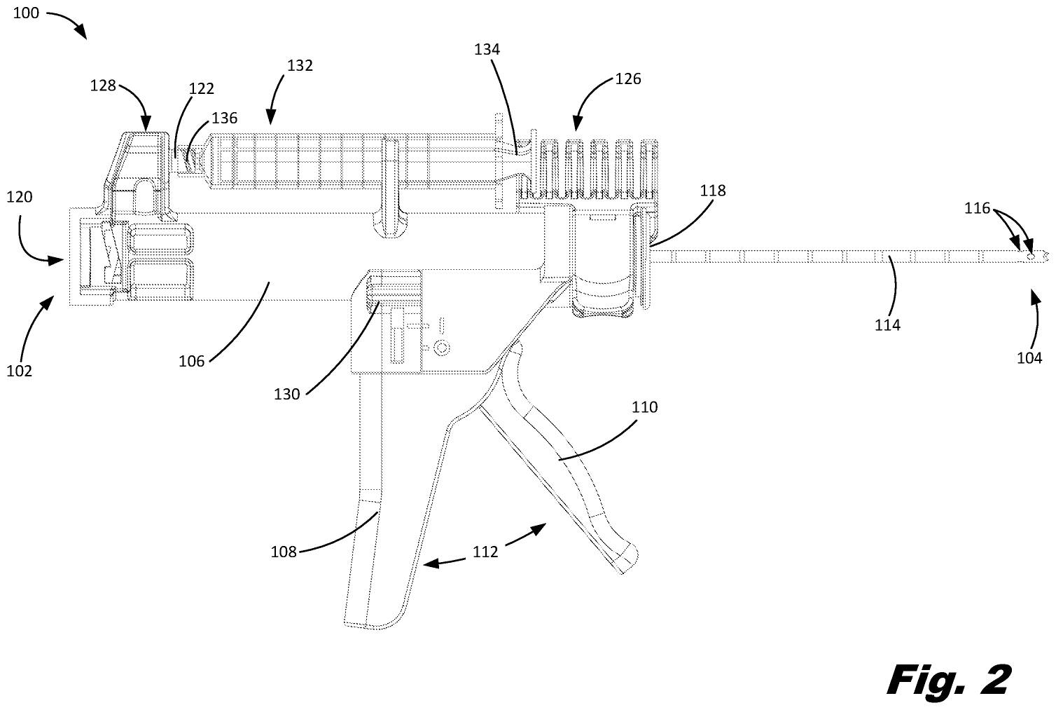

FIG. 2 illustrates a side view of a fluid aspiration device 100 including a removeable syringe according to an embodiment of the present disclosure. Again, the aspiration device 100 may include a proximal end 102 and a distal end 104 as described in connection with FIG. 1. The housing 106 of the aspiration device 100 may include a handle 108 and an actuator 110 extending o radially from a central longitudinal axis of the housing 106. The handle 108 and actuator 110 may be used by a user to actuate the actuator 110 in the actuator movement 112 direction in order to cause a ratcheting mechanism to concurrently advance the multi-slot plunger puller 126/patient contacting surface 118 distally while moving the aspiration device 100 proximally away from the body of a patient.

As described herein, the aspiration device 100, in an embodiment, may also include a plug 128 formed at a proximal end 102 of the aspiration device 100 where the fluidic path created by the cannula 114 meets with the fluid reservoir port 122. This plug 128 may be a permeable membrane made of a rubber or other self-healing material. The self-healing material that the plug 128 is made of may allow for the penetration of a needle into the plug 128 in order to introduce a substance into the collected fluid within the fluid reservoir. This substance may include a stabilizing chemical that preserved the fluid collected during use of the aspiration device 100.

The cannula 114 forms a fluidic channel, along with an aspiration cannula, between an extreme distal end of the cannula 114 to a fluid reservoir port 122. In the embodiment shown in FIG. 2, a removable syringe 132 has been fluidically coupled to the fluid reservoir port 122. In an embodiment, syringe threads 136 may be used to engage with complimentary threads formed on the fluid reservoir port 122 to secure the plunger 134 to the aspiration device 100. The fluid, during operation of the aspiration device 100, may be passed into the inner volume of the removable syringe 132 and act as the fluid reservoir described herein.

As described in FIG. 1, the actuation of the actuator 110 causes the multi-slot plunger puller 126 to draw the plunger 134 of the removable syringe 132 distally. As this occurs, the patient contacting surface 118 also moves distally. Additionally, as the multi-slot plunger puller 126 pulls the plunger 134 out form within the removable syringe 132, a negative pressure is created within the inner volume of the removable syringe 132. This negative pressure may draw a fluid such as bone marrow from within a bone, through the cannula 114 and an aspiration cannula, through the fluid reservoir port 122, and into the inner volume of the removable syringe 132.

In an embodiment, the aspiration device 100 may be used in a vertical position with the aspiration device 100 accessing a bone and bone marrow from above. In this embodiment, the distal end 104 of the cannula 114 may be inserted into the bone such that the cannula 114 is vertical. When the user actuates the actuator 110, the negative pressure created within the volume of the removable syringe 132 due to the multi-slot plunger puller 126 pulling the plunger 134 from within the removable syringe 132 and distally towards the patient, negative pressure may overcome the gravitational pull and drawn the bone marrow into the removable syringe 132. The gravitational pull of the aspirated bone marrow may also maintain the vacuum or negative pressure within the removable syringe 132.

The use of the removable syringe 132 may also facilitate the aspiration of bone marrow that exceeds the volumetric capacity of a single removable syringe 132. In this embodiment, when a first removable syringe 132 is decoupled from the fluid reservoir port 122, a second empty removable syringe 132 may be operatively coupled to the aspiration device 100 via the fluid reservoir port 122. During the replacement process, the aspiration device 100 may be maintained within the bone of the patient and gravity may help keep any aspirated bone marrow inside the full removable syringe 132 as it is being replaced. Still further, as the first removable syringe 132 is being replaced the user of the aspiration device 100 may grip the handle 108 and actuator 110 and, with the free hand, make the replacement.

As described herein, the aspiration device 100 may further include a ratcheting switch 130. The ratcheting switch 130 may engage the ratcheting mechanism, when in a first position, such that user-actuation of the actuator actuates the ratcheting mechanism formed in the housing 106. When the ratcheting switch 130 is in this first position, the multi-slot plunger puller 126 and the patient contacting surface 118 may be advanced distally when the actuator 110 is actuated by the user in the direction of the actuator movement 112 as indicated in FIG. 2. As described herein, the patient contacting surface 118 may be used to contact the surface of a patient's body when the cannula 114 of the aspiration device 100 is inserted into the patient's body and bone. As the patient contacting surface 118/multi-slot plunger puller 126 is advanced distally, the housing 106 may be moved proximally and the cannula 114 may also be retracted from within the patient's body and bone.

The ratcheting switch 130 may disengage the ratcheting mechanism, when the ratcheting switch is in a second position, such that the patient contacting surface 118 may move independent of the user-actuation of the ratcheting mechanism. Here, the disengagement of the ratcheting mechanism allows a user to disengage the multi-slot plunger puller 126 with a plunger of the syringe, move the multi-slot plunger puller 126 and the patient contacting surface 118, and reengage the multi-slot plunger puller 126 with the plunger in order to provide more moving distance for the plunger. In an embodiment, the selective advancement of the patient contacting surface 118 when the ratcheting switch 130 is in the second position may allow the user to bring the patient contacting surface 118 against the surface of the patient's body when the cannula 114 is sufficiently within the bone.

It is appreciated that when the aspiration device 100 is used to aspirate bone marrow from a patient's bone, the cannula 114 may be inserted into any bone within the human body. An advantageous bone to access with the cannula 114 of the aspiration device 100 may include the femur bone and the pelvic bone. However, it is appreciated that the cannula 114 may be inserted into any bone that contains an amount of bone marrow. As such, the distance between the patient's skin and the target bone may vary depending on the insertion location. The selective movement of the patient contacting surface 118 when the ratcheting switch 130 is placed in the second position accommodates for these varying depths.

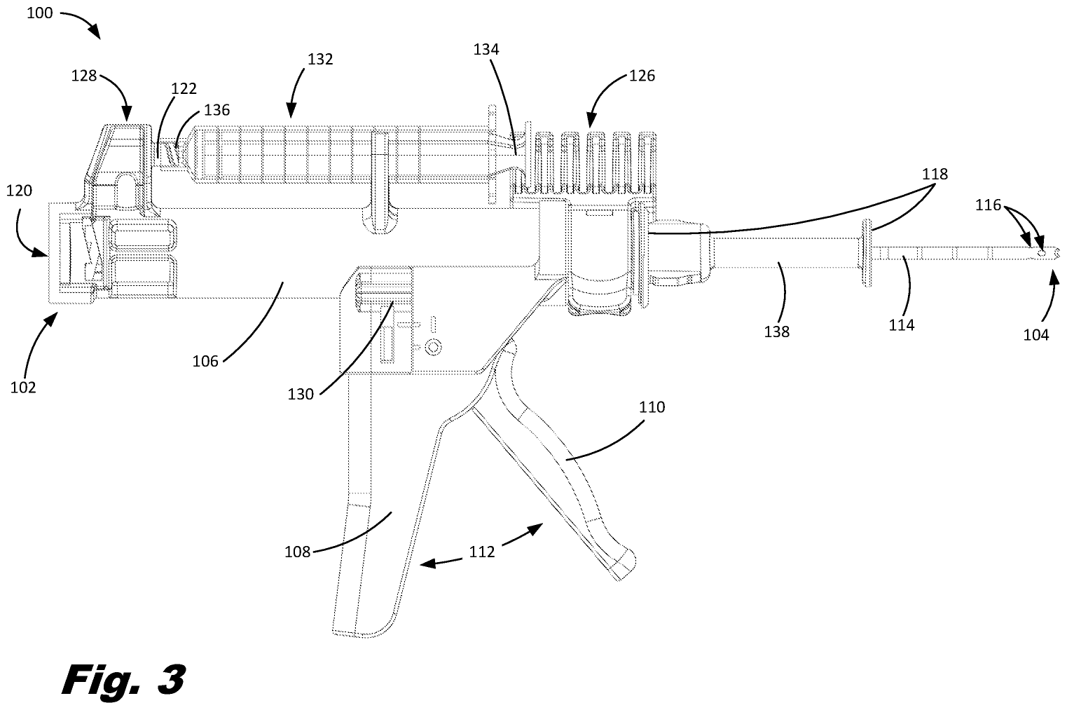

FIG. 3 illustrates a side view of a fluid aspiration device 100 including a removeable syringe and a depth stop 138 according to an embodiment of the present disclosure. Again, the aspiration device 100 may include a proximal end 102 and a distal end 104 as described in connection with FIG. 1. The housing 106 of the aspiration device 100 may include a handle 108 and an actuator 110 extending radially from a central longitudinal axis of the housing 106. The handle 108 and actuator 110 may be used by a user to actuate the actuator 110 in the actuator movement 112 direction in order to cause a ratcheting mechanism within the housing 106 to concurrently advance the multi-slot plunger puller 126/patient contacting surface 118 distally while moving the aspiration device 100 proximally away from the body of a patient.

As described herein, the aspiration device 100, in an embodiment, may also include a plug 128 formed at a proximal end 102 of the aspiration device 100 where the fluidic path created by the cannula 114 meets with the fluid reservoir port 122. This plug 128 may be a permeable membrane made of a rubber or other self-healing material. The self-healing material that the plug 128 is made of may allow for the penetration of a needle into the plug 128 in order to introduce a substance into the collected fluid within the fluid reservoir. This substance may include a stabilizing chemical that preserved the fluid collected during use of the aspiration device 100.

The cannula 114 forms a fluidic channel, along with an aspiration cannula, between an extreme distal end of the cannula 114 to a fluid reservoir port 122 to which a removable syringe 132 has been fluidically coupled to the fluid reservoir port 122. In an embodiment, syringe threads 136 may be used to engage with complimentary threads formed on the fluid reservoir port 122 to secure the plunger 134 to the aspiration device 100. It is appreciated that any type of coupled device may be used to operatively, fluidically couple the removable syringe 132 to the fluid reservoir port 122.

The fluid, during operation of the aspiration device 100, may be passed into the inner volume of the removable syringe 132 and act as the fluid reservoir described herein. In an embodiment, the removable syringe 132 may include measurement indicators that provide a visual indicator to the user the amount of fluid drawn into the removable syringe 132.

As described in FIGS. 1 and 2, the actuation of the actuator 110 causes the multi-slot plunger puller 126 to draw the plunger 134 of the removable syringe 132 distally. As this occurs, the patient contacting surface 118 also moves distally. Additionally, as the multi-slot plunger puller 126 pulls the plunger 134 out from within the removable syringe 132, a negative pressure is created within the inner volume of the removable syringe 132. This negative pressure may draw a fluid such as bone marrow from within a bone, through the cannula 114 and an aspiration cannula, through the fluid reservoir port 122, and into the inner volume of the removable syringe 132.

In an embodiment, the aspiration device 100 may be used in a vertical position with the aspiration device 100 accessing a bone and bone marrow from above. In this embodiment, the distal end 104 of the cannula 114 may be inserted into the bone such that the cannula 114 is vertical. When the user actuates the actuator 110, the negative pressure created within the volume of the removable syringe 132 due to the multi-slot plunger puller 126 pulling the plunger 134 from within the removable syringe 132 and distally towards the patient, negative pressure may overcome the gravitational pull and drawn the bone marrow into the removable syringe 132. The gravitational pull of the aspirated bone marrow may also maintain the vacuum or negative pressure within the removable syringe 132.

The use of the removable syringe 132 may also facilitate the aspiration of bone marrow that exceeds the volumetric capacity of a single removable syringe 132. In this embodiment, when a first removable syringe 132 is decoupled from the fluid reservoir port 122, a second empty removable syringe 132 may be operatively coupled to the aspiration device 100 via the fluid reservoir port 122. During the replacement process, the aspiration device 100 may be maintained within the bone of the patient and gravity may help keep any aspirated bone marrow inside the full removable syringe 132 as it is being replaced. Still further, as the first removable syringe 132 is being replaced the user of the aspiration device 100 may grip the handle 108 and actuator 110 and, with the free hand, make the replacement.

As described herein, the aspiration device 100 may further include a ratcheting switch 130. The ratcheting switch 130 may engage the ratcheting mechanism, when in a first position, such that user-actuation of the actuator actuates the ratcheting mechanism formed in the housing 106. When the ratcheting switch 130 is in this first position, the multi-slot plunger puller 126 and the patient contacting surface 118 may be advanced distally when the actuator 110 is actuated by the user in the direction of the actuator movement 112 as indicated in FIG. 2. As described herein, the patient contacting surface 118 may be used to contact the surface of a patient's body when the cannula 114 of the aspiration device 100 is inserted into the patient's body and bone. As the patient contacting surface 118/multi-slot plunger puller 126 is advanced distally, the housing 106 may be moved proximally and the cannula 114 may also be retracted from within the patient's body and bone.

The ratcheting switch 130 may disengage the ratcheting mechanism, when the ratcheting switch is in a second position, such that the patient contacting surface 118 may move independent of the user-actuation of the ratcheting mechanism. Here, the disengagement of the ratcheting mechanism allows a user to disengage the multi-slot plunger puller 126 with a plunger of the syringe, move the multi-slot plunger puller 126 and the patient contacting surface 118, and reengage the multi-slot plunger puller 126 with the plunger in order to provide more moving distance for the plunger. In an embodiment, the selective advancement of the patient contacting surface 118 when the ratcheting switch 130 is in the second position may allow the user to bring the patient contacting surface 118 against the surface of the patient's body when the cannula 114 is sufficiently within the bone.

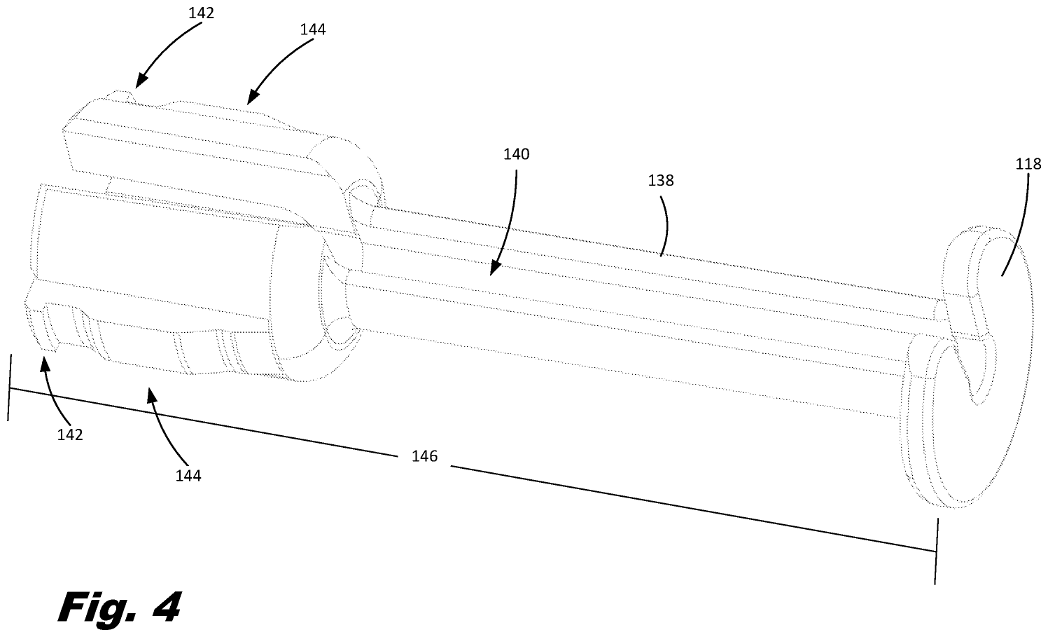

FIG. 3 shows us inclusion of a depth stop 138. As described herein, the depth stop 138 may be an extension added to a distal end of the aspiration device 100 where the patient contacting surface 118 contacts the patient in FIGS. 1 and 2. In this embodiment, the depth stop 138 creates a new patient contacting surface 118 that is distally further towards the patient than the patient contacting surface 118 as described and shown in FIG. 2, for example. As mentioned, the aspiration device 100 may be used to aspirate bone marrow from within a bone in the human body. Because this bone marrow may be extracted from any bone in the human body, the distance between the patient's skin and the bone may vary not only from patient to patient, but also by location along the surface of the patient's skin. For example, where bone marrow is to be removed from a pelvic bone, the insertion of the cannula 114 into the patient's body may alter the distance at which the cannula 114 can reach into the patient's body and through and into the bone. In relatively skinner patient's body, the distance may be significantly shorter than a relatively more overweight patient. Additionally, the distance between the skin of the patient and the pelvic bone may vary depending on whether the pelvic bone is accessed from the hip of the patient or from another location such as more anterior. With these varying depths to the bone, the depth stop 138 described in FIG. 3 may be part of a number of depth stops within a kit provided with the aspiration device 100. In this kit, each of the depth stops 138 provided may be of a different length to accommodate for the different depths to the bone among the different patients and aspiration locations. The depth stop 138 is described in more detail in FIG. 4 herein.

It is appreciated that when the aspiration device 100 is used to aspirate bone marrow from a patient's bone, the cannula 114 may be inserted into any bone within the human body. An advantageous bone to access with the cannula 114 of the aspiration device 100 may include the femur bone and the pelvic bone. However, it is appreciated that the cannula 114 may be inserted into any bone that contains an amount of bone marrow. As such, the distance between the patient's skin and the target bone may vary depending on the insertion location. The selective movement of the patient contacting surface 118 when the ratcheting switch 130 is placed in the second position accommodates for these varying depths.

FIG. 4 illustrates a side perspective view of a depth stop 138 according to an embodiment of the present disclosure. As described in FIG. 2, the depth stop 138 may be a separate piece operatively coupled to a patient contacting surface (e.g. 118, FIG. 1) to extend the location of the patient contacting surface 118 distally towards the patient. Again, as the depth of the bone relative to the patient's skin varies by location and patient, the depth stop 138 may be included or not to facilitate the aspiration of bone marrow with the aspiration device 100.

In an embodiment, the depth stop 138 may include a depth stop installation channel 140. The depth stop installation channel 140 may run the entire length of the depth stop 138 and allow for the depth stop 138 to be placed coaxially around the cannula (e.g., 114, FIG. 1) of the aspiration device 100. In an embodiment the width of the depth stop installation channel 140 may be slightly larger than the width of the cannula 114 in order to slide the depth stop 138 around the cannula 114. In an embodiment, the depth stop installation channel 140 is not present and installation of the depth stop 138 may include passing a distal end of the cannula 114 through a proximal end of the depth stop 138 until the distal end of the cannula 114 extends out from a distal end of the depth stop 138.

In an embodiment, the depth stop 138 includes one or more depth stop clips 142 and depth stop clipping surfaces 144 used to secure the depth stop 138 to the aspiration device 100 at the original patient contacting surface (e.g., 118 FIG. 1) on the aspiration device 100. The depth stop clips 142 may be moved radially towards a central axis of the depth stop 138 via a user pressing radially on the depth stop clipping surfaces 144. By doing so, the depth stop clips 142 can be inserted into a lip or shelf formed into the patient contacting surface 118 patient contacting surface (e.g., 118 FIG. 1) on the aspiration device 100 and clipped onto that surface. The depth stop 138 may be similarly removed by a user by actuating the depth stop clipping surfaces 144 sufficiently for the depth stop clips 142 to clear the lip or shelf surface.

As described herein, the depth stop length 146 may vary depending on the distance required between the patient contacting surface 118 and the bone of the patient. In an embodiment, the aspiration device 100 may include a depth stop kit that includes a plurality of depth stops 138 each having a different depth stop length 146. For example, the different depth stop lengths 146 of the plurality of depth stops 138 included in this kit may vary by quarter inches between neighboring sized depth stops 138.

It is appreciated that the operation of the aspiration device 100 described in connection with, at least, FIGS. 1-3 may include the use of the depth stop 138 or not. In some embodiments, the aspiration device 100 may be used without the depth stop 138 thereby relying on the patient contacting surface 118 as shown in FIG. 1 to contact the aspiration device 100 against the patient's skin. In an alternative embodiment, the aspiration device 100 may be used without any of the patient contacting surfaces 118 of the aspiration device 100 or depth stop 138 contacting the patient's skin. In this embodiment, the single-handed use of the aspiration device 100 may allow the user to steady the aspiration device 100 during the aspiration process described herein.

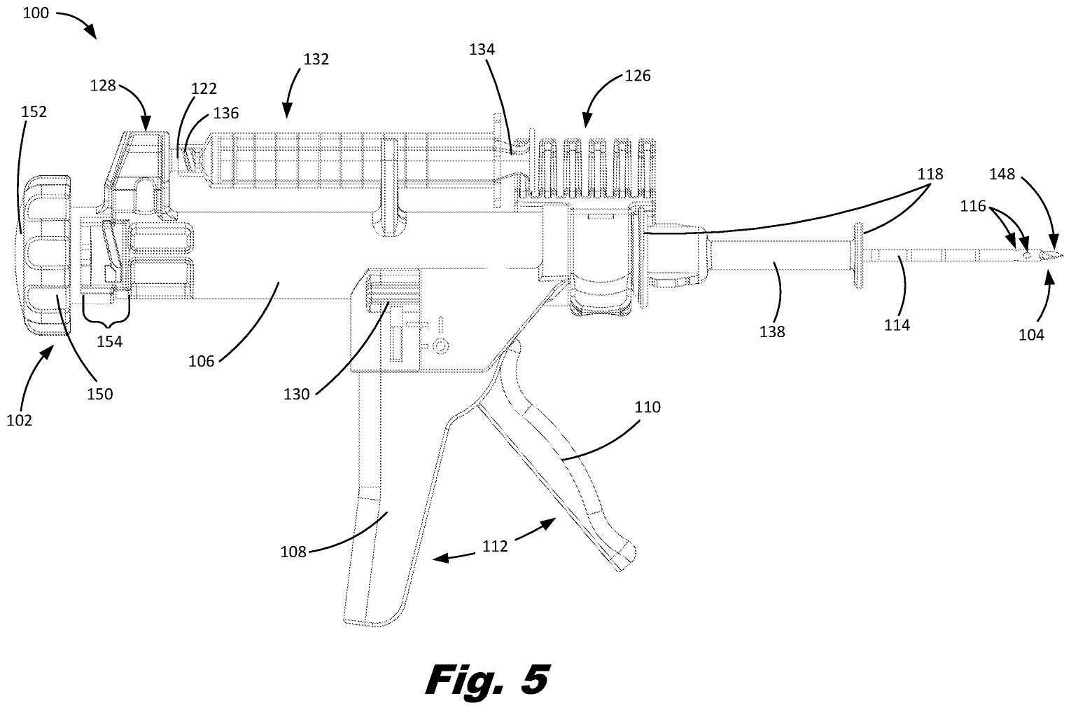

FIG. 5 illustrates a side view of a fluid aspiration device 100 including a removable syringe 132, a depth stop 138, and a sharp stylet according to an embodiment of the present disclosure. Again, the aspiration device 100 may include a proximal end 102 and a distal end 104 as described in connection with FIG. 1. The housing 106 of the aspiration device 100 may include a handle 108 and an actuator 110 extending radially from a central longitudinal axis of the housing 106. The handle 108 and actuator 110 may be used by a user to actuate the actuator 110 in the actuator movement 112 direction in order to cause a ratcheting mechanism within the housing 106 to concurrently advance the multi-slot plunger puller 126/patient contacting surface 118 distally while moving the aspiration device 100 proximally away from the body of a patient.

As described herein, the aspiration device 100, in an embodiment, may also include a plug 128 formed at a proximal end 102 of the aspiration device 100 where the fluidic path created by the cannula 114 meets with the fluid reservoir port 122. This plug 128 may be a permeable membrane made of a rubber or other self-healing material. The self-healing material that the plug 128 is made of may allow for the penetration of a needle into the plug 128 in order to introduce a substance into the collected fluid within the fluid reservoir. This substance may include a stabilizing chemical that preserved the fluid collected during use of the aspiration device 100.

The cannula 114 forms a fluidic channel, along with an aspiration cannula, between an extreme distal end of the cannula 114 to a fluid reservoir port 122 to which a removable syringe 132 has been fluidically coupled to the fluid reservoir port 122. In an embodiment, syringe threads 136 may be used to engage with complimentary threads formed on the fluid reservoir port 122 to secure the plunger 134 to the aspiration device 100. It is appreciated that any type of coupled device may be used to operatively, fluidically couple the removable syringe 132 to the fluid reservoir port 122.

The fluid, during operation of the aspiration device 100, may be passed into the inner volume of the removable syringe 132 and act as the fluid reservoir described herein. In an embodiment, the removable syringe 132 may include measurement indicators that provide a visual indicator to the user the amount of fluid drawn into the removable syringe 132.

As described in FIGS. 1-3, the actuation of the actuator 110 causes the multi-slot plunger puller 126 to draw the plunger 134 of the removable syringe 132 distally. As this occurs, the patient contacting surface 118 also moves distally. Additionally, as the multi-slot plunger puller 126 pulls the plunger 134 out from within the removable syringe 132, a negative pressure is created within the inner volume of the removable syringe 132. This negative pressure may draw a fluid such as bone marrow from within a bone, through the cannula 114 and an aspiration cannula, through the fluid reservoir port 122, and into the inner volume of the removable syringe 132.

The use of the removable syringe 132 may also facilitate the aspiration of bone marrow that exceeds the volumetric capacity of a single removable syringe 132. In this embodiment, when a first removable syringe 132 is decoupled from the fluid reservoir port 122, a second empty removable syringe 132 may be operatively coupled to the aspiration device 100 via the fluid reservoir port 122. During the replacement process, the aspiration device 100 may be maintained within the bone of the patient and gravity may help keep any aspirated bone marrow inside the full removable syringe 132 as it is being replaced. Still further, as the first removable syringe 132 is being replaced the user of the aspiration device 100 may grip the handle 108 and actuator 110 and, with the free hand, make the replacement.

As described herein, the aspiration device 100 may further include a ratcheting switch 130. The ratcheting switch 130 may engage the ratcheting mechanism, when in a first position, such that user-actuation of the actuator actuates the ratcheting mechanism formed in the housing 106. When the ratcheting switch 130 is in this first position, the multi-slot plunger puller 126 and the patient contacting surface 118 may be advanced distally when the actuator 110 is actuated by the user in the direction of the actuator movement 112 as indicated in FIG. 2. As described herein, the patient contacting surface 118 may be used to contact the surface of a patient's body when the cannula 114 of the aspiration device 100 is inserted into the patient's body and bone. As the patient contacting surface 118/multi-slot plunger puller 126 is advanced distally, the housing 106 may be moved proximally and the cannula 114 may also be retracted from within the patient's body and bone.

The ratcheting switch 130 may disengage the ratcheting mechanism, when the ratcheting switch is in a second position, such that the patient contacting surface 118 may move independent of the user-actuation of the ratcheting mechanism. Here, the disengagement of the ratcheting mechanism allows a user to disengage the multi-slot plunger puller 126 with a plunger of the syringe, move the multi-slot plunger puller 126 and the patient contacting surface 118, o and reengage the multi-slot plunger puller 126 with the plunger in order to provide more moving distance for the plunger. In an embodiment, the selective advancement of the patient contacting surface 118 when the ratcheting switch 130 is in the second position may allow the user to bring the patient contacting surface 118 against the surface of the patient's body when the cannula 114 is sufficiently within the bone.

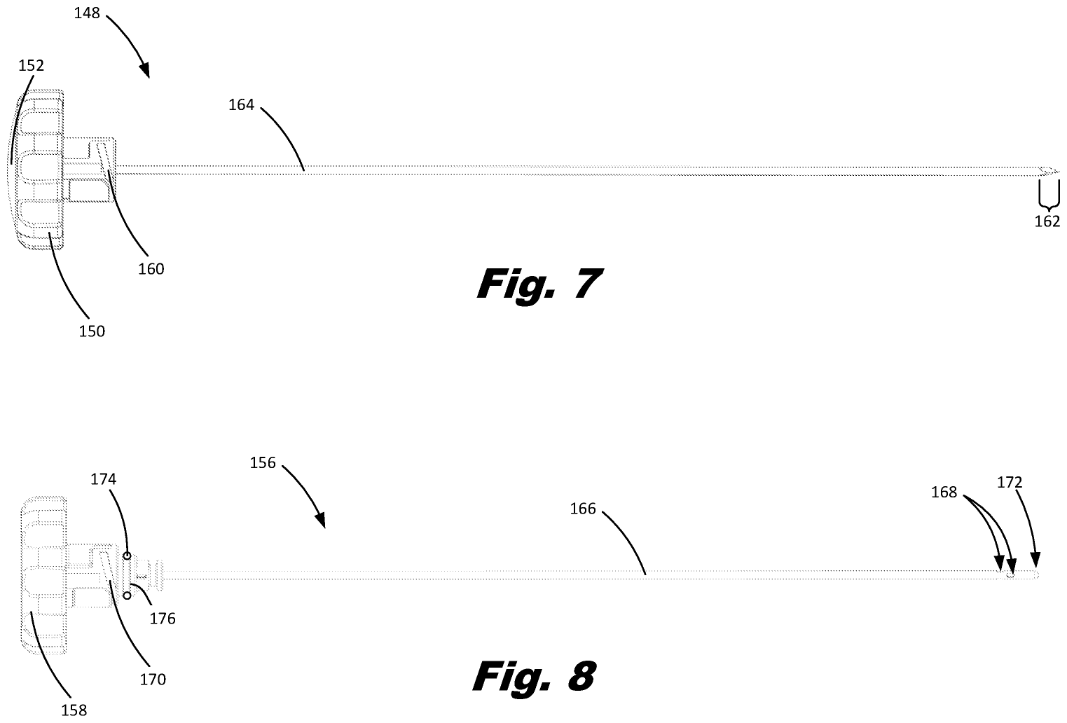

FIG. 5 further shows the coupling of a sharp stylet 148 to the aspiration device 100. The sharp stylet 148 may include a distal end where a sharpened end is used to penetrate a patient's tissue such as bone, muscle, fat, and the like. The sharp stylet 148 also includes a metal shaft passing, coaxially, through the inside of the cannula 114 and to a stylet knob 150 with its stylet impaction cap 152. The stylet knob 150 may include a locking mechanism 154 that interfaces with the proximal end 102 of the aspiration device 100 to securely engage the sharp stylet 148 with the aspiration device 100. Additional details of the sharp stylet 148 are described in reference to FIG. 7.

During use, the sharp stylet 148 may be inserted into the proximal end 102 of the aspiration device 100 and through the cannula 114. During installation, the locking mechanism 154 may lock the sharp stylet 148 into the aspiration device 100 and the extreme distal end of the sharp stylet 148 may extend out of the cannula 114. Because the distal end of the sharp stylet 148 is used to penetrate bone, the sharpened portion may be used to cut through and expand tissue in order to create a hole into these tissues. If and when necessary, such as when the sharp stylet 148 encounters the relatively harder bone tissues, the stylet impaction cap 152 may be used by the user to create an impaction force against the sharp stylet 148 and aspiration device 100 in order to force the sharp stylet 148 through the bone. In an embodiment, the user may use a hammer, for example, in order to create this impaction force against the stylet impaction cap 152.

In an embodiment, the locking mechanism 154 may be any type of mechanism that secures the sharp stylet 148 into the aspiration device 100. In the embodiment shown in FIG. 5, the locking mechanism 154 includes a thread or series of pegs that interface with locking threads formed into the proximal end of the aspiration device 100. In an embodiment, the locking mechanism 154 may include a detent used to provide tactile feedback to a user so that the user knows if and when the sharp stylet 148 is sufficiently seated into the threads formed on the proximal end of the aspiration device 100. As the locking mechanism 154 is completely seated, the distal end of the sharp stylet 148 extends past the distal end of the cannula 114.

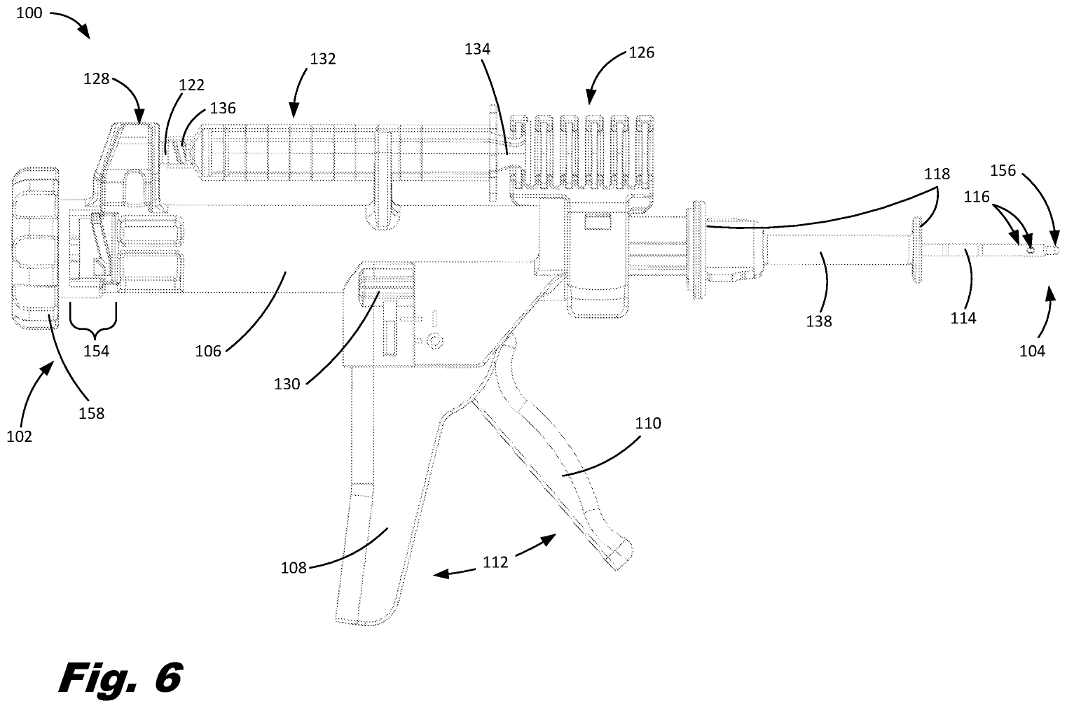

FIG. 6 illustrates a side view of a fluid aspiration device including a removable syringe 132, a depth stop 138, and an aspiration cannula 156 according to an embodiment of the present disclosure. Again, the aspiration device 100 may include a proximal end 102 and a distal end 104 as described in connection with FIG. 1. The housing 106 of the aspiration device 100 may include a handle 108 and an actuator 110 extending radially from a central longitudinal axis of the housing 106. The handle 108 and actuator 110 may be used by a user to actuate the actuator 110 in the actuator movement 112 direction in order to cause a ratcheting mechanism within the housing 106 to concurrently advance the multi-slot plunger puller 126/patient contacting surface 118 distally while moving the aspiration device 100 proximally away from the body of a patient.

As described herein, the aspiration device 100, in an embodiment, may also include a plug 128 formed at a proximal end 102 of the aspiration device 100 where the fluidic path created by the cannula 114 meets with the fluid reservoir port 122. This plug 128 may be a permeable membrane made of a rubber or other self-healing material. The self-healing material that the plug 128 is made of may allow for the penetration of a needle into the plug 128 in order to introduce a substance into the collected fluid within the fluid reservoir. This substance may include a stabilizing chemical that preserved the fluid collected during use of the aspiration device 100.

The cannula 114 forms a fluidic channel, along with an aspiration cannula, between an extreme distal end of the cannula 114 to a fluid reservoir port 122 to which a removable syringe 132 has been fluidically coupled to the fluid reservoir port 122. In an embodiment, syringe threads 136 may be used to engage with complimentary threads formed on the fluid reservoir port 122 to secure the plunger 134 to the aspiration device 100. It is appreciated that any type of coupled device may be used to operatively, fluidically couple the removable syringe 132 to the fluid reservoir port 122. As shown in FIG. 6, this fluidic channel also includes an aspiration cannula 156. The aspiration cannula 156 is described in more detail herein in connection with FIG. 8.

Installation of the aspiration cannula knob 158 may be similar to the installation of the sharp stylet 148 described in connection with FIG. 5. In this embodiment, the aspiration cannula knob 158 may be inserted into a proximal end 102 of the aspiration device 100 as indicated as an instrument port (e.g., 120, FIG. 1) in connection with FIG. 1. This instrument port may be used by the user to access an internal portion of the cannula 114 in order to allow the sharp stylet 148 and aspiration cannula 156 to be placed, coaxially within the cannula 114 during use of the aspiration device 100. In this embodiment, as the sharp stylet 148 is removed from within the cannula 114, the aspiration cannula 156 may be inserted, coaxially therein. The aspiration cannula knob 158 may, like the sharp stylet 148, include a number of pegs or threads that interface with threads of a locking mechanism 154. Again, as the pegs or threads formed on the aspiration cannula knob 158 are fully seated into the locking mechanism 154, a distal end of the aspiration cannula 156 extends past a distal end of the cannula 114.

In an embodiment, the aspiration cannula 156 includes one or more fenestrations (not shown in FIG. 6). In an embodiment, as the aspiration cannula 156 is fully seated within the cannula 114, the fenestrations of the aspiration cannula 156 may match up with the cannula fenestrations 116 of the cannula 114. By aligning these two fenestrations in the canula 114 and the aspiration cannula 156, the aspiration device 100 may be able to draw fluids such as bone marrow into the cannula, into the aspiration cannula 156, through the aspiration cannula 156/cannula 114 and into the removable syringe 132 via the fluid reservoir port 122.

As described herein the fluid (e.g., bone marrow), during operation of the aspiration device 100, may be passed into the inner volume of the removable syringe 132 and act as the fluid reservoir described herein. In an embodiment, the removable syringe 132 may include measurement indicators that provide a visual indicator to the user the amount of fluid drawn into the removable syringe 132.

As described in FIGS. 1-3, the actuation of the actuator 110 causes the multi-slot plunger puller 126 to draw the plunger 134 of the removable syringe 132 distally. As this occurs, the patient contacting surface 118 also moves distally. Additionally, as the multi-slot plunger puller 126 pulls the plunger 134 out from within the removable syringe 132, a negative pressure is created within the inner volume of the removable syringe 132. This negative pressure may draw a fluid such as bone marrow from within a bone, through the cannula 114/aspiration cannula 156, through the fluid reservoir port 122, and into the inner volume of the removable syringe 132.

The use of the removable syringe 132 may also facilitate the aspiration of bone marrow that exceeds the volumetric capacity of a single removable syringe 132. In this embodiment, when a first removable syringe 132 is decoupled from the fluid reservoir port 122, a second empty removable syringe 132 may be operatively coupled to the aspiration device 100 via the fluid reservoir port 122. During the replacement process, the aspiration device 100 may be maintained within the bone of the patient and gravity may help keep nay aspirated bone marrow inside the full removable syringe 132 as it is being replaced. Still further, as the first removable syringe 132 is being replaced the user of the aspiration device 100 may grip the handle 108 and actuator 110 and, with the free hand, make the replacement.

As described herein, the aspiration device 100 may further include a ratcheting switch 130. The ratcheting switch 130 may engage the ratcheting mechanism, when in a first position, such that user-actuation of the actuator actuates the ratcheting mechanism formed in the housing 106. When the ratcheting switch 130 is in this first position, the multi-slot plunger puller 126 and the patient contacting surface 118 may be advanced distally when the actuator 110 is actuated by the user in the direction of the actuator movement 112 as indicated in FIG. 2. As described herein, the patient contacting surface 118 may be used to contact the surface of a patient's body when the cannula 114 of the aspiration device 100 is inserted into the patient's body and bone. As the patient contacting surface 118/multi-slot plunger puller 126 is advanced distally, the housing 106 may be moved proximally and the cannula 114 may also be retracted from within the patient's body and bone.

The ratcheting switch 130 may disengage the ratcheting mechanism, when the ratcheting switch is in a second position, such that the patient contacting surface 118 may move independent of the user-actuation of the ratcheting mechanism. Here, the disengagement of the ratcheting mechanism allows a user to disengage the multi-slot plunger puller 126 with a plunger of the syringe, move the multi-slot plunger puller 126 and the patient contacting surface 118, and reengage the multi-slot plunger puller 126 with the plunger in order to provide more moving distance for the plunger. In an embodiment, the selective advancement of the patient contacting surface 118 when the ratcheting switch 130 is in the second position may allow the user to bring the patient contacting surface 118 against the surface of the patient's body when the cannula 114 is sufficiently within the bone.

FIG. 7 illustrates a side view of a sharp stylet 148 according to an embodiment of the present disclosure. As described herein, the sharp stylet 148 may include a sharp stylet shaft 164 with a sharp distal end 162 formed at a distal end of the sharp stylet 148 and a stylet knob 150 and stylet impaction cap 152 formed at a proximal end of the sharp stylet 148.

The sharp distal end 162 may be of any length sufficient to allow for penetration of the sharp stylet 148 into a tissue such as muscle, fat, and bone as described herein. In an embodiment, the sharp distal end 162 may be formed to create a hole, the diameter of the sharp stylet shaft 164, into a bone in order to give access to the cannula (e.g., 114, FIG., 1) of the aspiration device 100 as described herein.

The sharp stylet 148 further includes a stylet knob 150 used by a user to grip the sharp stylet 148 and insert the sharp stylet 148, coaxially, into the cannula of the aspiration device 100. The stylet knob 150 may have any surfaces that allow the user to properly grip the proximal end of the sharp stylet 148 in order to insert and, in the present embodiments, twist the sharp stylet 148 so that stylet locking threads 160 formed on the stylet knob 150 can engage with a locking mechanism (e.g., 154, FIGS. 5 and 6) formed into the proximal end of the aspiration device 100.

During use and when the sharp stylet 148 is inserted into the cannula and locking into the housing of the aspiration device 100, the user may insert the aspiration device 100 into a patient's body allowing the sharp distal end 162 to pierce and cleave muscle tissue, fat tissue, and bone tissue apart to create the hole into the interior of a bone. In an embodiment, where the sharp distal end 162 encounters the bone tissue, a relatively more significant amount of force may be necessary to insert the sharp distal end 162 of the sharp stylet 148 into the bone and bone marrow. As such, the stylet knob 150 may include a stylet impaction cap 152 that receives impact force from an object such as a hammer or other weighted device. With this impact force placed on the stylet impaction cap 152, the distal end of the sharp stylet 148 with its sharp distal end 162 may penetrate the bone and pass into the bone marrow.

FIG. 8 illustrates a side view of an aspiration cannula 156 according to an embodiment of the present disclosure. The aspiration cannula 156 may include an aspiration cannula shaft 166 with an aspiration cannula capped end 172 at a distal end of the aspiration cannula 156 and an aspiration cannula knob 158 formed at a proximal end of the aspiration cannula shaft 166. The aspiration cannula knob 158 may be used by a user to insert the aspiration cannula 156, coaxially, into the cannula of the aspiration device 100. The aspiration cannula knob 158 may have any surfaces that allow the user to properly grip the proximal end of the aspiration cannula 156 in order to insert and, in the present embodiments, twist the aspiration cannula knob 158 so that aspiration cannula locking threads 170 formed on the aspiration cannula knob 158 can engage with a locking mechanism (e.g., 154, FIGS. 5 and 6) formed into the proximal end of the aspiration device 100.

During use and when the sharp stylet 148 has been removed and the aspiration cannula 156 has been inserted into the cannula and locked into the housing of the aspiration device 100, the user may actuate the actuator (e.g., 110, FIGS. 1-5) in the actuator movement 112 direction in order to cause a ratcheting mechanism formed in the housing (e.g., 106, FIG. 1) of the aspiration device 100 to concurrently advance the multi-slot plunger puller (e.g., 126, FIG. 1)/patient contacting surface (e.g., 118, FIG. 1) distally while moving the aspiration device 100 proximally away from the body of a patient. As this occurs, the negative pressure formed by the pulling of the plunger (e.g., 134, FIG. 2) from within the syringe (e.g., 132, FIG. 2) causes bone marrow to be drawn into the aspiration cannula fenestrations 168 formed in the aspiration cannula 156 as well as those through the cannula fenestrations (e.g., 116, FIG. 1). As described herein, as the aspiration cannula locking threads 170 are properly seated within the locking mechanism (e.g.,154, FIGS. 5 and 6), the aspiration cannula fenestrations 168 and cannula fenestrations 116 may align so that bone marrow may pass into the fluidic channel formed by the cannula (e.g., 114, FIG. 1)/aspiration cannula 156, fluid reservoir port (e.g., 122, FIG. 1), and the syringe as described herein.

The aspiration cannula 156 may also include an aspiration cannula capped end 172. This aspiration cannula capped end 172 may cause aspiration of the bone marrow through the cannula fenestrations 116 and aspiration cannula fenestrations 168. In an embodiment, the aspiration cannula capped end 172 may not be present and the distal end of the aspiration cannula 156 may be open allowing for the bone marrow to be aspirated into the distal end of the aspiration cannula 156 as well as the fenestrations.

In an embodiment, the aspiration cannula 156 may further include an O-ring 174. The O-ring 174 (shown in a cross section) may be formed in an O-ring slot 176. Because the aspiration cannula 156 is used to aspirate bone marrow and other fluids from the body in order to perform medical procedures on and with that bone marrow, the O-ring 174 may be used to isolate the bone marrow from any external bacteria. The O-ring 174 being placed in O-ring slot 176 may prevent bone marrow from exiting the aspiration device 100 while also maintaining the negative pressure (e.g., vacuum) created in the syringe during operation of the aspiration device 100. Although the aspiration cannula 156 shows the use of a single O-ring 174, the present specification contemplates the use of multiple O-rings 174 as well.

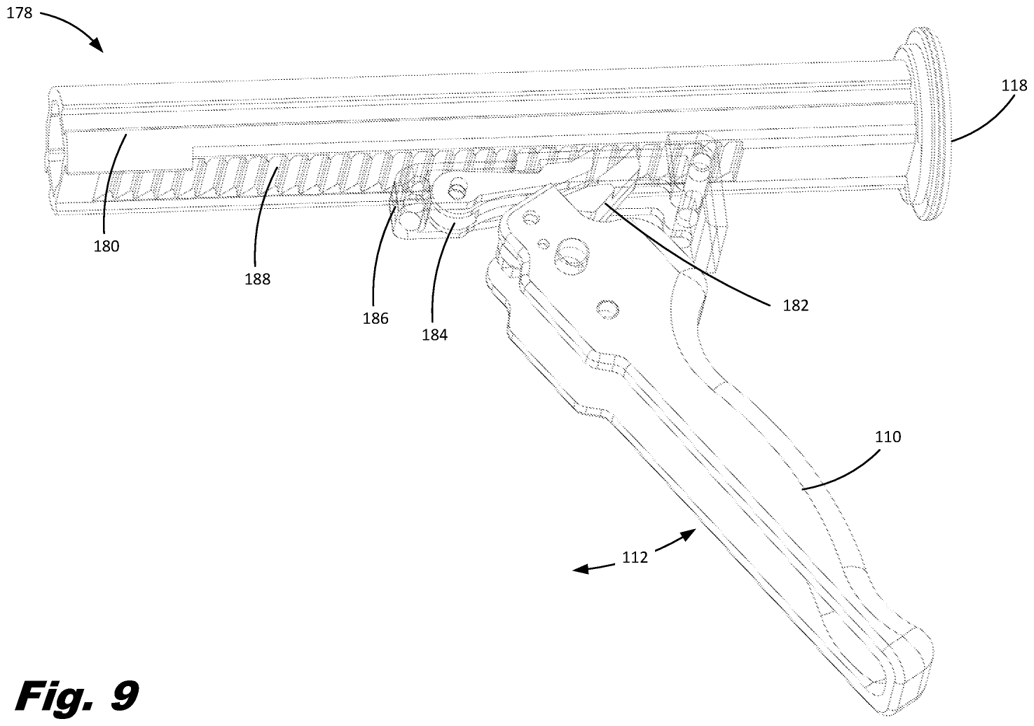

FIG. 9 illustrates a side perspective view of a ratcheting mechanism 178 of the fluid aspiration device 100 according to an embodiment of the present disclosure. As shown in FIG. 9, the ratcheting mechanism 178 may include a shuttle 180 that is mechanically coupled to the actuator 110 via a pushing pawl 182, a holding pawl 184, and a releasing arm 186 (shown in ghost to show the holding pawl 184). Each of these and their respective operations are discussed herein.

The shuttle 180 may be made of any type of material that can withstand the mechanical stresses of pushing and pulling a cannula, with a sharp stylet 148 or aspiration cannula 156 or not, into and out of a patient's body. The shuttle 180 may be formed coaxially within the housing 106 of the aspiration device 100 and may be formed around the cannula 114 such that the cannula is placed along a central, longitudinal axis of the shuttle 180. At a bottom surface of the shuttle 180, a plurality of ratcheting teeth 188 may be formed such that a pushing pawl 182 and a holding pawl 184 may engage with the ratcheting teeth 188. The rise and tread of the ratcheting teeth 188 may provide for the incremental advancement of the shuttle 180 when the user actuates the actuator 110. In an embodiment, this incremental advancement may be set such that the advancement of the shuttle 180 within the housing and the movement of a multi-slot plunger puller 126 operatively coupled to the shuttle 180 causes a specific amount of fluid to be drawn into the removeable syringe (e.g., 132, FIG. 2). In an example, the incremental advancement may cause a single cubic centimeter (1 ml) of fluid or bone marrow to be aspirated from the bone.

In the embodiment shown in FIG. 9, the pushing pawl 182 may be operatively coupled to the actuator 110 via any pin or connecting device that allows the actuator 110 and pushing pawl 182 to hinge at that connection point. As the actuator 110 is actuated by the user, the pushing pawl 182 may push against one of the ratcheting teeth 188 thereby moving the shuttle 180 a distance as described herein. Once the actuator 110 has been actuated in the actuator movement 112 direction towards a handle, the pushing pawl 182 may push against one of the ratcheting teeth 188 while a holding pawl 184 stays in place and falls behind another of the ratcheting teeth 188 along the bottom surface of the shuttle 180. When the user returns the actuator 110 to its pre-actuation position, the holding pawl 184 may maintain the location of the shuttle 180 as it was incremented by the pushing pawl 182. The holding pawl 184 may specifically force the shuttle 180 and its patient contacting surface 118 against the patient. In this embodiment, because force is presented against the patient contacting surface 118 by the patient's skin as the cannula is concurrently being removed from the patient via actuation of the actuator 110, the holding pawl 184 may counter that force by securing the shuttle 180 against this force. In an embodiment, the holding pawl 184 may be operatively coupled to the housing of the aspiration device 100 via a pin or other connection device such that the holding pawl 184 may rotate radially away from the shuttle 180. In an embodiment, the holding pawl 184 may include a spring or other biasing device that maintains an upper portion of the holding pawl 184 against the ratcheting teeth 188.

The ratcheting mechanism 178 may further include a releasing arm 186. The releasing arm 186 may be operatively coupled, at a distal location, to the housing using a pin or other coupling device such that the releasing arm 186 may hinge at this contact point radially away from and towards the shuttle 180. At a proximal end of the releasing arm 186, the releasing arm 186 may be operatively coupled, via a pin or other coupling device, to a ratcheting switch (e.g., 130, FIG. 1). The ratcheting switch 130 may engage the holding pawl 184 such that when the ratcheting switch 130 is in a first position, user-actuation of the actuator 110 actuates the ratcheting mechanism 178 and causes the shuttle 180 to move distally along with the coupled multi-slot plunger puller 126. The ratcheting switch 130 may disengage the ratcheting mechanism 178 or otherwise allow the shuttle 180 to move proximally, when the ratcheting switch 130 is in a second position. This causes the patient contacting surface 118 to move independent of the user-actuation of the ratcheting mechanism and either advanced distally or proximally within the housing of the aspiration device 100. Here, the disengagement of the ratcheting mechanism allows a user to disengage the multi-slot plunger puller 126 with a plunger of the syringe, move the multi-slot plunger puller 126/shuttle 180 with its patient contacting surface 118, and reengage the multi-slot plunger puller 126 with the plunger in order to provide more moving distance for the plunger as described herein.

As described herein in connection with FIGS. 1-8, the handle 108 and actuator 110 may be used by a user to actuate the actuator 110 in the actuator movement 112 direction in order to cause a ratcheting mechanism to concurrently advance the multi-slot plunger puller 126/patient contacting surface 118/ shuttle 180 distally while moving the aspiration device 100 proximally away from the body of a patient. When the canula 114 has been inserted into the bone to aspirate bone marrow, the cannula may concurrently be pulled out from within the bone the distance that the housing 106 of the aspiration device 100 is moved proximally. This may cause the cannula 114/aspiration cannula 156 to not be entirely removed from within the bone but instead move the cannula 114/aspiration cannula 156 to a different location within the bone in order to aspirate additional bone marrow from within the bone. This process may continue until the cannula 114/aspiration cannula 156 is fully drawn out of the bone. A user may know when the cannula 114/aspiration cannula 156 is fully drawn out of the bone when bone marrow is no longer seen by the user to be aspirated into the removeable syringe 132. In this example, the removeable syringe 132 may be clear with delineated volume markings made on the outer surface of the removeable syringe 132. This allows the user to observe when bone marrow is no longer being aspirated and instead other fluids such as blood is being aspirated instead. At this point, the user may withdraw the cannula 114/aspiration cannula 156 from within the patient's body and address whether enough bone marrow has been aspirated. If not, a new insertion site on the patient's body may be identified and the process may continue with the user reinserting the sharp stylet 148 into the cannula 114 and reinserting the cannula 114/sharp stylet 148 into the patient's body.

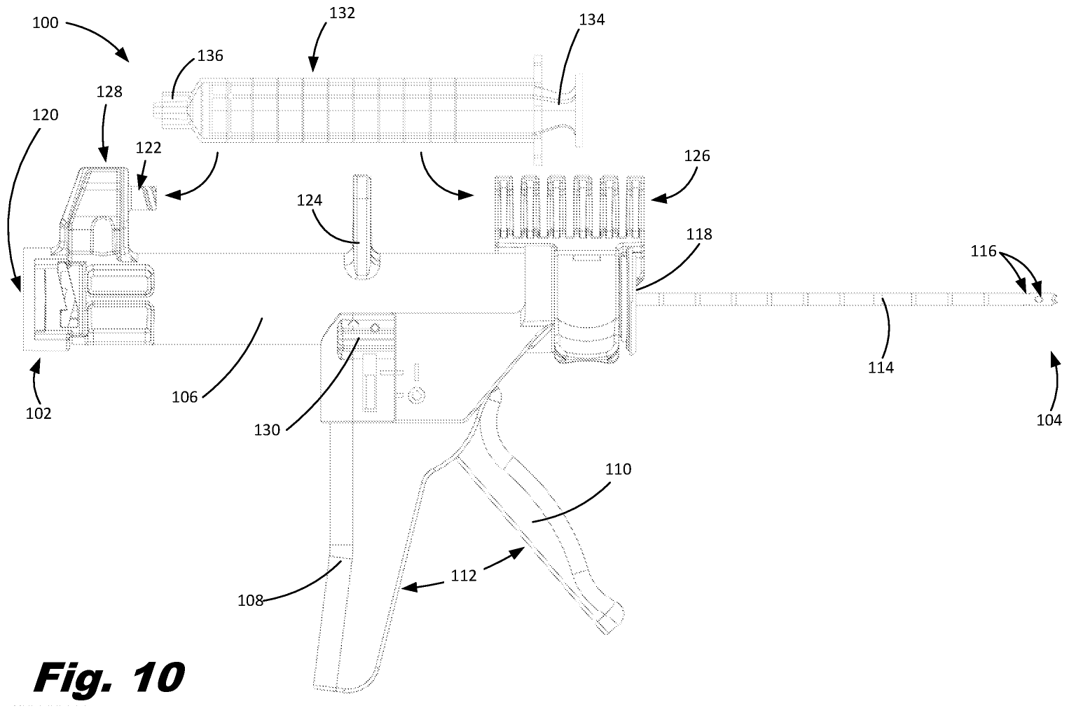

FIGS. 10-16 describe an embodiment method of using the aspiration device 100. These are meant to only show an example use of the aspiration device 100 and the present specification contemplates that the order of some operations may be swapped without going beyond the description herein. FIG. 10 illustrates a side view of a fluid aspiration device 100 with a syringe 132 to be operatively coupled to the fluid aspiration device 100 according to an embodiment of the present disclosure. As described herein, the fluid reservoir may be a removable syringe 132 in an example. The use of the removable syringe 132 allows for the aspiration device 100 to create a negative pressure within the removable syringe 132 as the multi-slot plunger puller 126 pulls the plunger 134 from within the removable syringe 132. The present specification, however, contemplates the use of other fluid reservoirs.

FIG. 10, as with other figures herein, shows the aspiration device 100 may include a proximal end 102 and a distal end 104 as described in connection with FIG. 1. The housing 106 of the aspiration device 100 may include a handle 108 and an actuator 110 extending radially from a central longitudinal axis of the housing 106. The handle 108 and actuator 110 may be used by a user to actuate the actuator 110 in the actuator movement 112 direction in order to cause a ratcheting mechanism within the housing 106 to concurrently advance the multi-slot plunger puller 126/patient contacting surface 118 distally while moving the aspiration device 100 proximally away from the body of a patient. In an embodiment, the aspiration device 100 may also include a plug 128 formed at a proximal end 102 of the aspiration device 100 where the fluidic path created by the cannula 114 meets with the fluid reservoir port 122. The cannula 114 forms a fluidic channel, along with an aspiration cannula, between an extreme distal end of the cannula 114 to a fluid reservoir port 122 to which a removable syringe 132 has been fluidically coupled to the fluid reservoir port 122.

As described herein, the aspiration device 100 may further include a ratcheting switch 130. The ratcheting switch 130 may engage the ratcheting mechanism, when in a first position, such that user-actuation of the actuator actuates the ratcheting mechanism formed in the housing 106. When the ratcheting switch 130 is in this first position, the multi-slot plunger puller 126 and the patient contacting surface 118 may be advanced distally when the actuator 110 is actuated by the user in the direction of the actuator movement 112 as indicated in FIG. 2. As described herein, the patient contacting surface 118 may be used to contact the surface of a patient's body when the cannula 114 of the aspiration device 100 is inserted into the patient's body and bone. As the patient contacting surface 118/multi-slot plunger puller 126 is advanced distally, the housing 106 may be moved proximally and the cannula 114 may also be retracted from within the patient's body and bone.

The ratcheting switch 130 may disengage the ratcheting mechanism, when the ratcheting switch is in a second position, such that the patient contacting surface 118 may move independent of the user-actuation of the ratcheting mechanism. Here, the disengagement of the ratcheting mechanism allows a user to disengage the multi-slot plunger puller 126 with a plunger of the syringe, move the multi-slot plunger puller 126 and the patient contacting surface 118, and reengage the multi-slot plunger puller 126 with the plunger in order to provide more moving distance for the plunger. In an embodiment, the selective advancement of the patient contacting surface 118 when the ratcheting switch 130 is in the second position may allow the user to bring the patient contacting surface 118 against the surface of the patient's body when the cannula 114 is sufficiently within the bone. In the context of FIG. 10, the user may place the ratcheting switch 130 in the first position to prevent movement of the patient contacting surface 118, the multi-slot plunger puller 126, and the shuttle 180 as described herein.

FIG. 10 shows how a user may couple the removable syringe 132 to the aspiration device 100. In an embodiment, the user may move a proximal end of the removable syringe 132 to interface with the fluid reservoir port 122, secure the removable syringe 132 to the fluid reservoir port 122 via the syringe threads 136 and corresponding threads on the fluid reservoir port 122, and set the removable syringe 132 into the fluid reservoir mount 124.

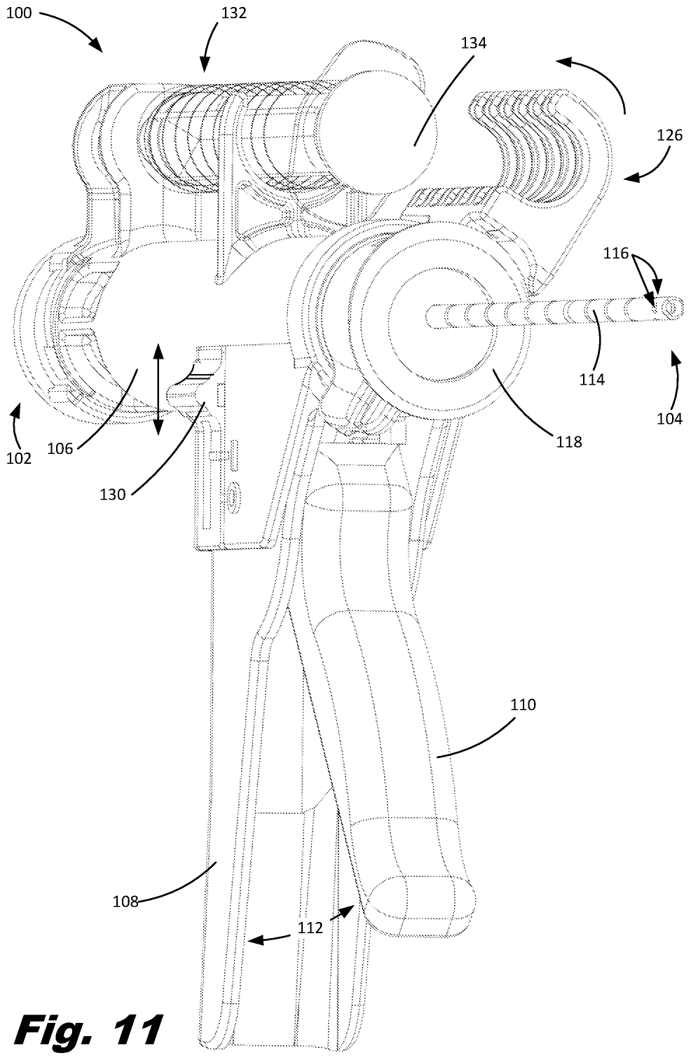

FIG. 11 illustrates a front perspective view of a fluid aspiration device 100 with the removable syringe 132 operatively coupled thereto according to an embodiment of the present disclosure. FIG. 11 shows the removable syringe 132 in place and fluidically coupled to the aspiration device 100. In this embodiment, the ratcheting switch 130 is in the first position thereby preventing movement of the patient contacting surface 118/shuttle 180/multi-slot plunger puller 126 distally along the cannula 114. This may also prevent the patient contacting surface 118/shuttle 180/multi-slot plunger puller 126 from moving proximally 102 as well.

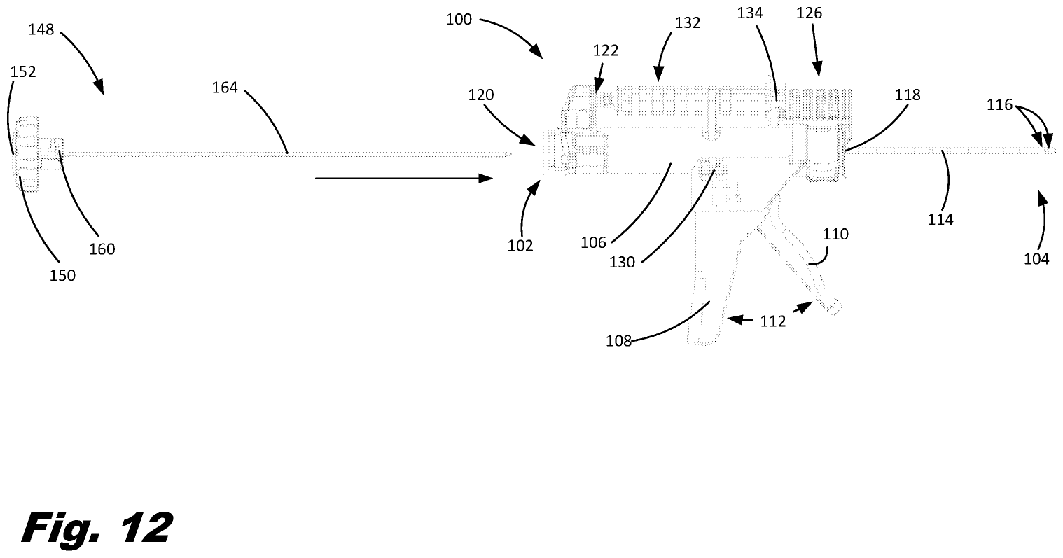

In FIG. 11, a user may move the multi-slot plunger puller 126 as indicated to engage with the plunger 134. The multi-slot plunger puller 126 may include a number of slots that may allow the multi-slot plunger puller 126 to interface with the plunger 134 such that the plunger 134 is only moved into and out of the removeable syringe 132 via movement of the multi-slot plunger puller 126 as described herein. Having engaged the multi-slot plunger puller 126 with the plunger 134 the user may move onto the processes described in connection with FIG. 12.

FIG. 12 illustrates a side view of a fluid aspiration device 100 depicting the introduction of a sharp stylet 148 into the fluid aspiration device 100 according to an embodiment of the present disclosure. As described herein, the aspiration device 100 may include an instrument port 120 into which one of the sharp stylet 148 or aspiration cannula 156 may be inserted. In the embodiment shown in FIG. 12, the sharp stylet 148 is inserted into the instrument port 120 and into the cannula 114 to place the sharp stylet 148 in a coaxial position within the cannula 114.

The sharp stylet 148 may include a distal end where a sharpened end is used to penetrate a patient's tissue such as bone, muscle, fat, and the like. The sharp stylet 148 also includes a metal shaft passing, coaxially, through the inside of the cannula 114 and to a stylet knob 150 with its stylet impaction cap 152. The stylet knob 150 may include a locking mechanism 154 that interfaces with the proximal end 102 of the aspiration device 100 to securely engage the sharp stylet 148 with the aspiration device 100. Additional details of the sharp stylet 148 are described in reference to FIG. 7.

During use, the sharp stylet 148 may be inserted into the proximal end 102 of the aspiration device 100 and through the cannula 114. During installation, a locking mechanism 154 may lock the sharp stylet 148 into the aspiration device 100 and the extreme distal end of the sharp stylet 148 may extend out of the cannula 114. Because the distal end of the sharp stylet 148 is used to penetrate bone, the sharpened portion may be used to cut through and expand tissue in order to create a hole into these tissues. If and when necessary, such as when the sharp stylet 148 encounters the relatively harder bone tissues, the stylet impaction cap 152 may be used by the user to create an impaction force against the sharp stylet 148 and aspiration device 100 in order to force the sharp stylet 148 through the bone. In an embodiment, the user may use a hammer, for example, in order to create this impaction force against the stylet impaction cap 152. In an embodiment, the locking mechanism 154 may be any type of mechanism that secures the sharp stylet 148 into the aspiration device 100. In the embodiment shown in FIG. 5, the locking mechanism 154 includes a thread or series of pegs that interface with locking threads formed into the proximal end of the aspiration device 100. In an embodiment, the locking mechanism 154 may include a detent used to provide tactile feedback to a user so that the user knows if and when the sharp stylet 148 is sufficiently seated into the threads formed on the proximal end of the aspiration device 100. As the locking mechanism 154 is completely seated, the distal end of the sharp stylet 148 extends past the distal end of the cannula 114.

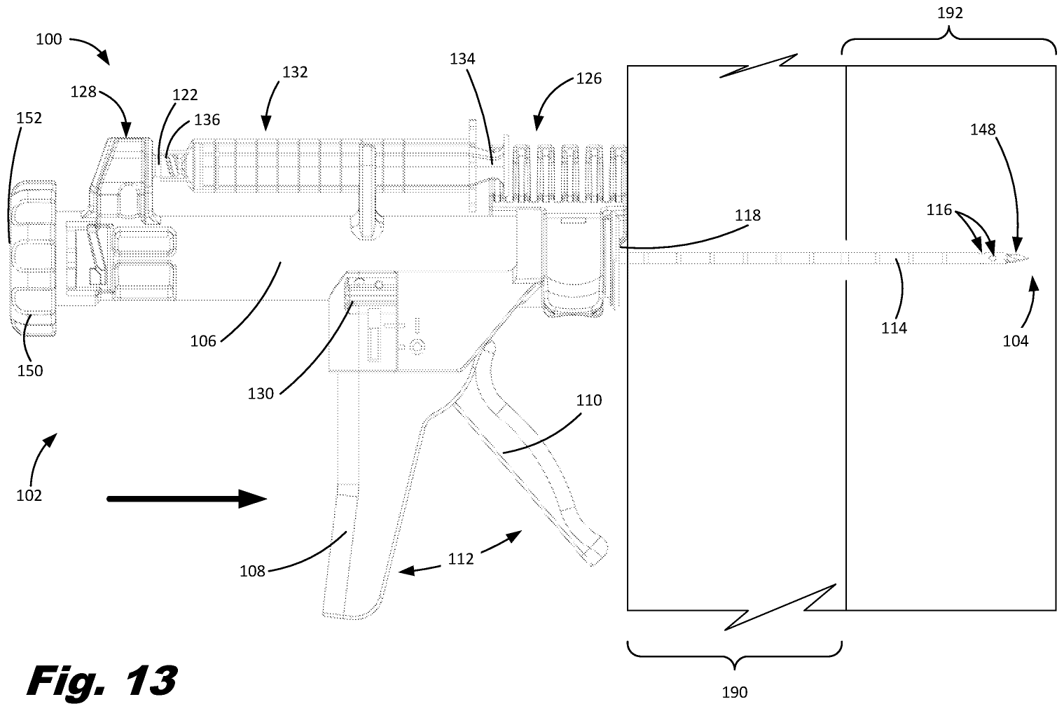

FIG. 13 illustrates a side view of a fluid aspiration device 100 with a removable syringe 132 and a sharp stylet 148 being inserted into body tissue 190 and bone 192 according to an embodiment of the present disclosure. The body tissue 190 may include a first layer that may include one of a muscle tissue, skin tissue, among other tissues. The bone 192 may be any bone tissue within the human body.

FIG. 13 shows the insertion of the sharp stylet 148 and cannula 114 into the body tissue 190 and through the bone 192 to access, in an example embodiment, bone marrow. The depth of the cannula 114/sharp stylet 148 into the bone may be measure using whether the patient contacting surface 118 is contacting the skin of the user. In the embodiment in FIG. 13, the patient contacting surface 118 is contacting the skin of the patient. However, the present specification contemplates that the cannula 114/sharp stylet 148 may be inserted into the bone without the patient contacting surface 118 contacting the patient's skin allowing the user to freely hold the aspiration device 100 steady during aspiration.

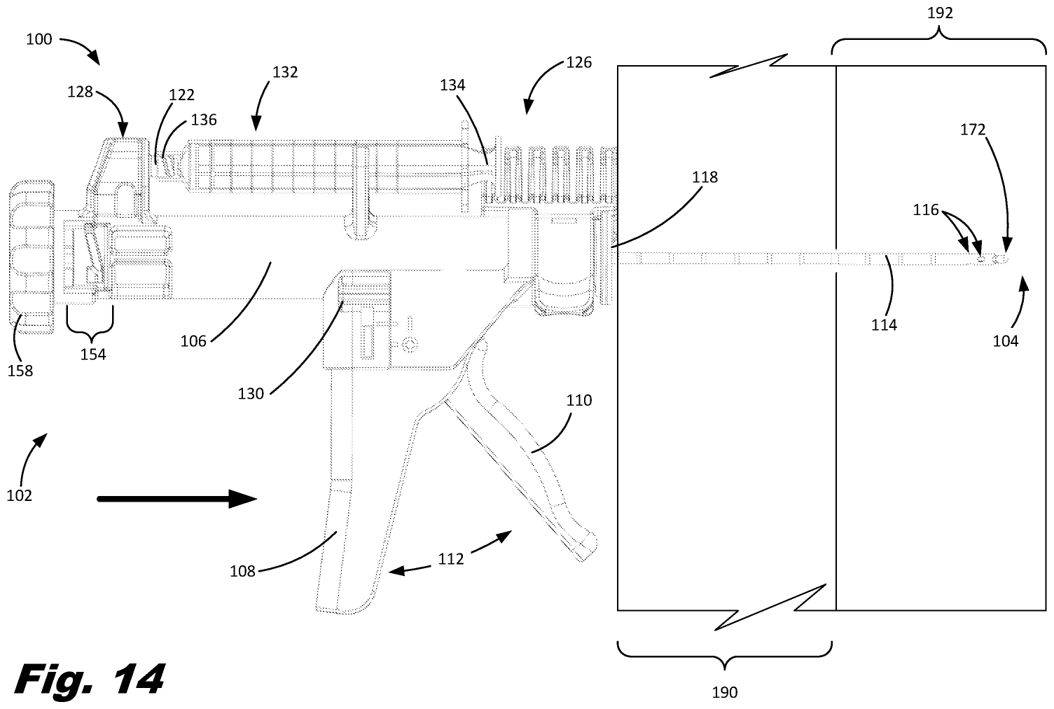

FIG. 13 further shows that the actuator 110 has not been actuated in the actuator movement 112 due to the multi-slot plunger puller 126 not being extended distally from the housing 106 of the actuator 110. At this point, the actuation of the actuator 110 may not aspirate the bone marrow due to the sharp stylet 148 being in place. However, replacement of the sharp stylet 148 with the aspiration cannula 156 may allow for aspiration of the bone marrow. FIG. 14 describes such a replacement.

FIG. 14 illustrates a side view of an aspiration device 100 with a removable syringe 132 and an aspiration cannula 156 being inserted into a body tissue 190 and bone 192 according to an embodiment of the present disclosure. As described herein, the sharp stylet 148 may be replaced with the aspiration cannula 156 after the sharp stylet 148/cannula 114 have reached the bone 192 and is accessing the bone marrow therein. In order to switch the sharp stylet 148 with the aspiration cannula 156 the user may hold the aspiration device 100 steady as the user unlocks the stylet locking threads 160 from the locking mechanism 154 as described herein. The aspiration cannula 156 may be then placed within the instrument port 120 and coaxially into the cannula 114. Again, the user may lock the aspiration cannula 156 to the housing 106 using the aspiration cannula locking threads 170 to engage the locking mechanism 154. Once fully seated an aspiration cannula capped end 172 may extend distally out of the distal end of the cannula 114. Additionally, in an embodiment, when the aspiration cannula 156 is fully seated and locked into the housing 106 of the aspiration device 100, one or more aspiration cannula fenestrations 168 will align with one or more cannula fenestrations 116.

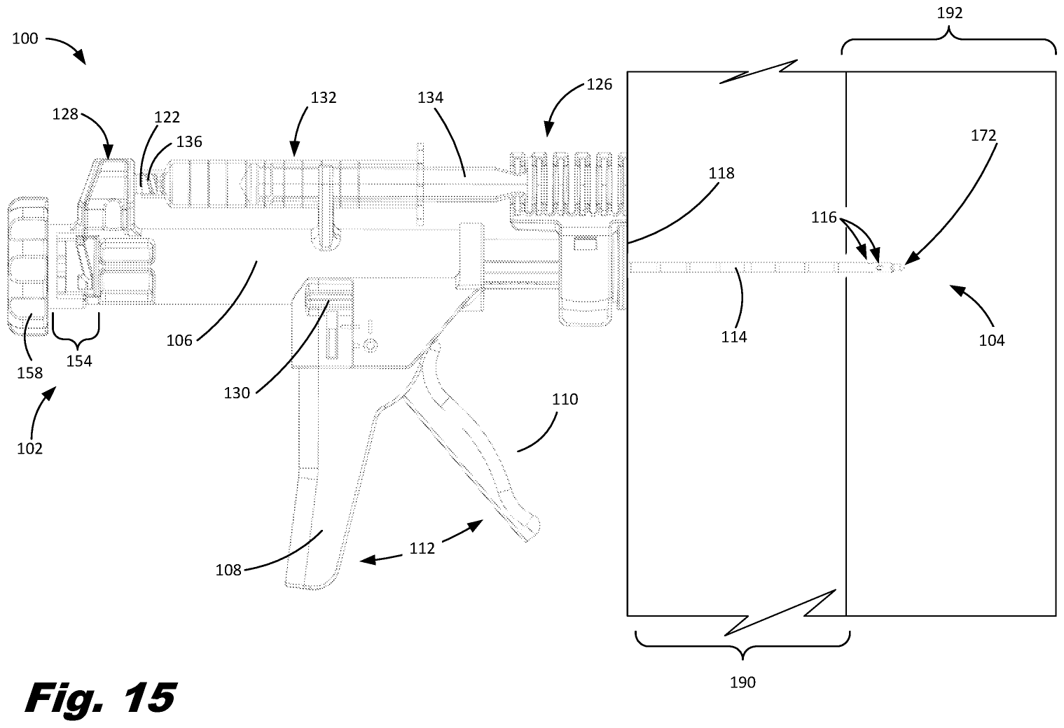

FIG. 14 shows the aspiration device 100 now ready for aspiration of the bone marrow from the bone 192. A user may do this by actuating the actuator 110 as shown in FIG. 15. FIG. 15 illustrates a side view of an aspiration device 100 with a removable syringe 132 and an aspiration cannula 156 being inserted into a tissue and resulting actuation of a ratcheting mechanism 178 according to an embodiment of the present disclosure. As described in connection with FIG. 9, the ratcheting mechanism 178 is formed into the housing 106 of the aspiration device 100 such that the cannula 114 is coaxially placed within the ratcheting mechanism 178 along a central, longitudinal axis of the shuttle 180.

Actuation of the actuator 110 may cause a number of devices of the aspiration device 100 to perform those processes described herein. As the actuator 110 is actuated by the user, the pushing pawl 182 may push against one of the ratcheting teeth 188 thereby moving the shuttle 180 a distance as described herein. Once the actuator 110 has been actuated in the actuator movement 112 direction towards a handle, the pushing pawl 182 may push against one of the ratcheting teeth 188 while a holding pawl 184 stays in place and falls behind another of the ratcheting teeth 188 along the bottom surface of the shuttle 180. When the user returns the actuator 110 to its pre-actuation position, the holding pawl 184 may maintain the location of the shuttle 180 as it was incremented by the pushing pawl 182. The holding pawl 184 may specifically force the shuttle 180 and its patient contacting surface 118 against the patient.

Concurrently as the shuttle 180 and its patient contacting surface 118 against the patient, the multi-slot plunger puller 126, being operatively coupled to the shuttle, is moved distally pressing against the patient's skin. As this occurs, the plunger 134 of the removable syringe 132 is also pulled distally. As this occurs, a negative pressure or vacuum is created within the removable syringe 132 and an amount of fluid such as the bone marrow in the bone 192 is aspirated out of the bone 192, into the cannula fenestrations 116 and aspiration cannula fenestrations 168, through the aspiration cannula 156, past the fluid reservoir port 122 and into the volume created in the removable syringe 132.

Additionally, the movement of the patient contacting surface 118 distally causes the aspiration device 100 to move proximally towards the user. Because the canula 114 is coupled to the aspiration device 100 and moves independent respective to the shuttle 180, the cannula is moved a distance out of the bone 192. FIG. 15 shows that the distal end of the cannula 114 and aspiration cannula 156 are not yet pulled fully out of the bone 192. This allows the user to actuate the actuator 110 and drawn another portion of bone marrow into the removable syringe 132 from a different location within the bone 192. This process may continue until the user observes that other fluids such as blood is being aspirated instead indicating that the aspiration cannula 156 has existed the bone 192 and is no longer accessing bone marrow for aspiration. At this point, the user may withdraw the cannula 114/aspiration cannula 156 from within the patient's body and address whether enough bone marrow has been aspirated. If not, a new insertion site on the patient's body may be identified and the process may continue with the user reinserting the sharp stylet 148 into the cannula 114 and reinserting the cannula 114/sharp stylet 148 into the patient's body.

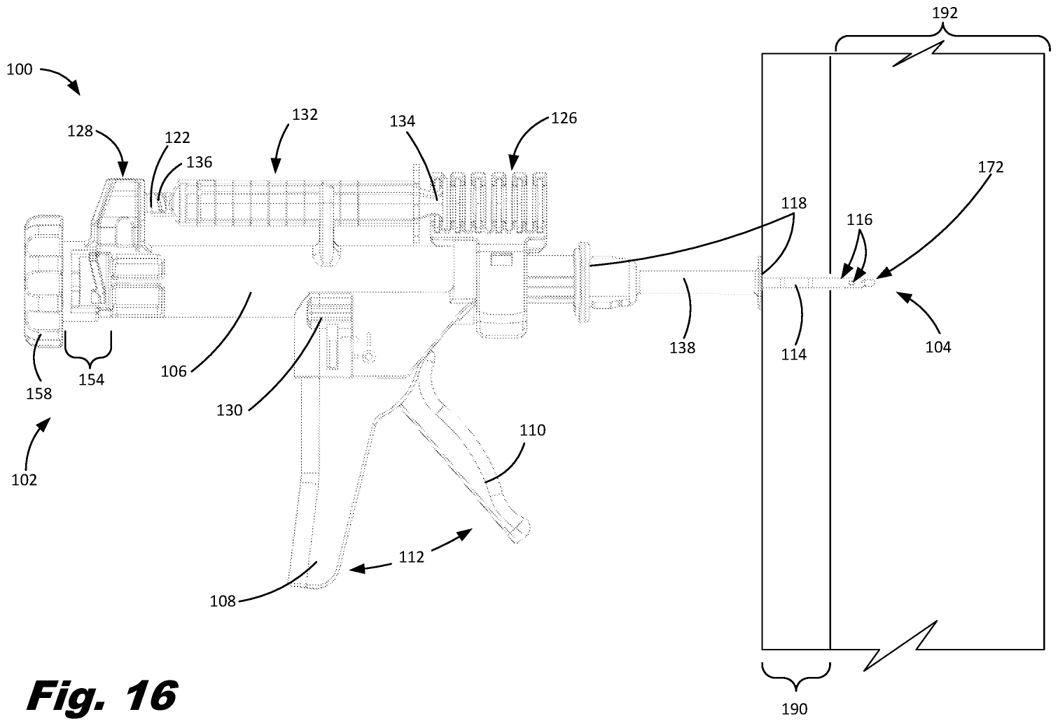

FIG. 16 illustrates a side view of an aspiration device 100 with a removable syringe 132, an aspiration cannula 156, and a depth stop 138 being inserted into a tissue 190 and bone 192 according to an embodiment of the present disclosure. As described herein, the depth stop 138 may be an extension added to a distal end of the aspiration device 100 where the patient contacting surface 118 contacts the patient in FIGS. 1 and 2. In this embodiment, the depth stop 138 creates a new patient contacting surface 118 that is distally further towards the patient than the patient contacting surface 118 as described and shown in FIG. 2, for example. As mentioned, the aspiration device 100 may be used to aspirate bone marrow from within a bone in the human body. Because this bone marrow may be extracted from any bone in the human body, the distance between the patient's skin and the bone may vary not only from patient to patient, but also by location along the surface of the patient's skin. For example, where bone marrow is to be removed from a pelvic bone, the insertion of the cannula 114 into the patient's body may alter the distance at which the cannula 114 can reach into the patient's body and through and into the bone. In relatively skinner patient's body, the distance may be significantly shorter than a relatively more overweight patient. Additionally, the distance between the skin of the patient and the pelvic bone may vary depending on whether the pelvic bone is accessed from the hip of the patient or from another location such as more anterior. With these varying depths to the bone, the depth stop 138 described in FIG. 3 may be part of a number of depth stops within a kit provided with the aspiration device 100. In this kit, each of the depth stops 138 provided may be of a different length to accommodate for the different depths to the bone among the different patients and aspiration locations.

In an alternative embodiment to implementing the depth stop 138, the user may disengage the ratcheting mechanism, when the ratcheting switch is in a second position, such that the patient contacting surface 118 may move independent of the user-actuation of the ratcheting mechanism and be placed against the skin of the patient after the cannula 114 has accessed the bone marrow. Here, the disengagement of the ratcheting mechanism allows a user to disengage the multi-slot plunger puller 126 with a plunger of the syringe, move the multi-slot plunger puller 126 and the patient contacting surface 118, and reengage the multi-slot plunger puller 126 with the plunger in order to provide more moving distance for the plunger.

The steps, process, methods and aspects of the operation of the embodiments herein and discussed herein need not be performed in any given or specified order. It is contemplated that additional blocks, steps, or functions may be added, some blocks, steps or functions may not be performed, blocks, steps, or functions may occur contemporaneously, and blocks, steps or functions from one flow diagram may be performed within another flow diagram.

The aspiration device and method of use described herein may provide for a bone marrow aspiration device that is compact and can be operated, during aspiration, with one hand by a user. The orientation of the removable syringe 132 relative to the housing 106 of the aspiration device 100 may allow gravity to help in the aspiration process as well as allow for a user to swap out a first removable syringe 132 for a replacement removable syringe 132 when the first removable syringe 132 is full. Because a vacuum is created within the removable syringe 132 when the plunger 134 is pulled by the multi-slot plunger puller 126, the bone marrow may be aspirated in a more efficient way. Additionally, the operation of the aspiration device 100 may allow for a single device to be used to penetrate through the tissues and access bone marrow while also swapping out a sharp stylet 148 with an aspiration cannula 156 to aspirate the bone marrow.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. §112 Para. 6. It will be apparent to those having skill in the art that changes may be made to the details of the above-described embodiments without departing from the underlying principles of the invention.

While specific embodiments and applications of the present invention have been illustrated and described, it is to be understood that the invention is not limited to the precise configuration and components disclosed herein. Various modifications, changes, and variations which will be apparent to those skilled in the art may be made in the arrangement, operation, and details of the methods and systems of the present invention disclosed herein without departing from the spirit and scope of the invention.

What is claimed is:

1. A bone marrow aspiration device comprising:
a housing having a proximal end and a distal end;
a cannula, insertable into a tissue, extending from the distal end;
a ratcheting mechanism coupled to the housing;
a reservoir configured to receive a fluid; and
an actuator operatively coupled to the ratcheting mechanism and to a distal surface of the bone marrow aspiration device such that user-actuation of the actuator actuates the ratcheting mechanism to draw fluid through the cannula, into the housing, and into the reservoir, and retracts the cannula relative to the distal surface.

2. The bone marrow aspiration device of claim 1, wherein the reservoir comprises a syringe with a plunger.

3. The bone marrow aspiration device of claim 2, further comprising:
a multi-slot plunger puller operatively and selectively coupled to the plunger of the syringe;
wherein user-actuation of the actuator causes the multi-slot plunger puller to move distally, creating a negative pressure within the syringe and drawing the fluid into the syringe.

4. The bone marrow aspiration device of claim 1, further comprising:
a sharp stylet that is insertable into the cannula to allow the distal end of the cannula to penetrate through the tissue.

5. The bone marrow aspiration device of claim 1, further comprising an aspiration cannula that is insertable into the cannula, the aspiration cannula comprising:
aspiration cannula fenestrations that align with cannula fenestrations formed in the cannula when the aspiration cannula is correctly seated, coaxially, within the cannula.

6. The bone marrow aspiration device of claim 5, wherein the aspiration cannula comprises an O-ring that fluidically seals the aspiration cannula against an inner surface of the housing to create a sealed fluidic path along the aspiration cannula and to the reservoir.

7. The bone marrow aspiration device of claim 1, further comprising a ratcheting switch that is operable to:
engage the ratcheting mechanism such that user-actuation of the actuator actuates the ratcheting mechanism when the ratcheting switch is in a first position, wherein, with the engagement of the ratcheting mechanism and user-actuation of the actuator, the ratcheting mechanism moves the distal surface distally along the cannula; and
disengage the ratcheting mechanism when the ratcheting switch is in a second position such that the distal surface may move independently of the user-actuation of the ratcheting mechanism.

8. The bone marrow aspiration device of claim 1, further comprising:
a depth stop extension operatively fixed to the distal surface to abut a surface of a tissue and, during a percutaneous incision of the cannula into the tissue, maintain a distance between the surface of the tissue and the distal end.

9. An aspiration device, comprising:
a housing having a proximal end and a distal end;
a cannula, insertable into a tissue, extending from the distal end;
a ratcheting mechanism coupled to the housing; and
an actuator operatively coupled to the ratcheting mechanism such that user-actuation of the actuator actuates the ratcheting mechanism to draw fluid through the cannula, into the housing, and into a removeable syringe fluidically coupled to a proximal end of the cannula.

10. The aspiration device of claim 9, further comprising:
a multi-slot plunger puller operatively and selectively coupled to a plunger of the removeable syringe;
wherein user-actuation of the actuator causes the multi-slot plunger puller to move distally, creating a negative pressure within the syringe and drawing the fluid into the removeable syringe.

11. The aspiration device of claim 9, further comprising an aspiration cannula that is insertable into the cannula, the aspiration cannula comprising:
aspiration cannula fenestrations that align with cannula fenestrations formed in the cannula when the aspiration cannula is correctly seated, coaxially, within the cannula.

12. The aspiration device of claim 11, further comprising a reservoir configured to receive the fluid;
wherein the aspiration cannula comprises an O-ring that fluidically seals the aspiration cannula against an inner surface of the housing and creates a sealed fluidic path along the aspiration cannula and to the reservoir.

13. The aspiration device of claim 9, further comprising a ratcheting switch that is operable to:
engage the ratcheting mechanism such that user-actuation of the actuator actuates the ratcheting mechanism when the ratcheting switch is in a first position; and
disengage the ratcheting mechanism when the ratcheting switch is in a second position such that a distal surface of the aspiration device may move independently of the user-actuation of the ratcheting mechanism.

14. The aspiration device of claim 9, further comprising:
a depth stop extension operatively fixed to a distal surface of the aspiration device to abut a surface of a tissue and, during a percutaneous incision of the cannula into the tissue, maintain a distance between the surface of the tissue and the distal end.

15. A bone marrow aspiration device comprising:
a housing having a proximal end and a distal end;
a cannula, insertable into a tissue, extending from the distal end;
a removeable syringe fluidically coupled to a proximal end of the cannula at a proximal end of the housing; and
an actuator operatively coupled to the syringe such that user-actuation of the actuator draws bone marrow through the cannula, through the housing, and into a proximal end of the removeable syringe.

16. The bone marrow aspiration device of claim 15, further comprising:
a multi-slot plunger puller operatively and selectively coupled to a plunger of the removeable syringe;
wherein user-actuation of the actuator causes the multi-slot plunger puller to move distally, creating a negative pressure within the syringe and drawing the bone marrow into the removeable syringe.

17. The bone marrow aspiration device of claim 15, further comprising a ratcheting switch that is operable to:
engage a ratcheting mechanism such that user-actuation of the actuator actuates the ratcheting mechanism when the ratcheting switch is in a first position; and
disengage the ratcheting mechanism when the ratcheting switch is in a second position such that a distal surface of the bone marrow aspiration device may move independent of the user-actuation of the ratcheting mechanism.

18. The bone marrow aspiration device of claim 15, further comprising:
a depth stop extension operatively fixed to a distal surface of the bone marrow aspiration device to abut a surface of a tissue and, during a percutaneous incision of the cannula into the tissue, maintain a distance between the surface of the tissue and the distal end.

19. The bone marrow aspiration device of claim 15, further comprising:
a sharp stylet that is insertable into the cannula to allow the distal end of the cannula to penetrate through the tissue.

20. The bone marrow aspiration device of claim 15, further comprising an aspiration cannula that is insertable into the cannula, the aspiration cannula comprising:
aspiration cannula fenestrations that align with cannula fenestrations formed in the cannula when the aspiration cannula is correctly seated, coaxially, within the cannula.
